SEQUENCE LISTING (27 sequences)

SEQ ID NO: 1 - protein, 232 aa, Homo sapiens
  VARIANT (5)...(5): Xaa = Cys or Ser
EPKSXDKTHTCPPCPAPEXXGXPSVFLFPPKPKDTLMISRTPEVTCVVVDVSHEDPEVKFNWYVDGVEVHNAKTKPREEQYNSTYRVVSVLTVLHQDWLNGKEYKCKVSNKALPXXIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 2368 bp, Homo sapiens
  CDS (23)...(1759)
ctcggggctcccgagggacgccatgcggggaggcaggggcgcccctttctggctgtggccgctgcccaagctggcgctgctgcctctgttgtgggtgcttttccagcggacgcgtccccagggcagcgccgggccactgcagtgctacggagttggacccttgggcgacttgaactgctcgtgggagcctcttggggacctgggagccccctccgagttacacctccagagccaaaagtaccgttccaacaaaacccagactgtggcagtggcagccggacggagctgggtggccattcctcgggaacagctcaccatgtctgacaaactccttgtctggggcactaaggcaggccagcctctctggccccccgtcttcgtgaacctagaaacccaaatgaagccaaacgccccccggctgggccctgacgtggacttttccgaggatgaccccctggaggccactgtccattgggccccacctacatggccatctcataaagttctgatctgccagttccactaccgaagatgtcaggaggcggcctggaccctgctggaaccggagctgaagaccatacccctgacccctgttgagatccaagatttggagctagccactggctacaaagtgtatggccgctgccggatggagaaagaagaggatttgtggggcgagtggagccccattttgtccttccagacaccgccttctgctccaaaagatgtgtgggtatcagggaacctctgtgggacgcctggaggagaggaacctttgcttctatggaaggccccagggccctgtgtgcaggtgagctacaaagtctggttctgggttggaggtcgtgagctgagtccagaaggaattacctgctgctgctccctaattcccagtggggcggagtgggccagggtgtccgctgtcaacgccacaagctgggagcctctcaccaacctctctttggtctgcttggattcagcctctgccccccgtagcgtggcagtcagcagcatcgctgggagcacggagctactggtgacctggcaaccggggcctggggaaccactggagcatgtagtggactgggctcgagatggggaccccctggagaaactcaactgggtccggcttccccctgggaacctcagtgctctgttaccagggaatttcactgtcggggtcccctatcgaatcactgtgaccgcagtctctgcttcaggcttggcctctgcatcctccgtctgggggttcagggaggaattagcacccctagtggggccaacgctttggcgactccaagatgcccctccagggacccccgccatagcgtggggagaggtcccaaggcaccagcttcgaggccacctcacccactacaccttgtgtgcacagagtggaaccagcccctccgtctgcatgaatgtgagtggcaacacacagagtgtcaccctgcctgaccttccttggggtccctgtgagctgtgggtgacagcatctaccatcgctggacagggccctcctggtcccatcctccggcttcatctaccagataacaccctgaggtggaaagttctgccgggcatcctattcttgtggggcttgttcctgttggggtgtggcctgagcctggccacctctggaaggtgctaccacctaaggcacaaagtactgccccgctgggtctgggagaaagttcctgatcctgccaacagcagttcaggccttctggggccccccaggccacaggttctggcctgaaccacacgtctggctgggggctgccagccaggctagagggatgctcatgcaggttgcaccccagtcctggattagccctcttgatggatgaagacactgaggactcagagaggctgagtcacttacctgaggacacccagccaggcagagctgggattgaaggacccctatagagaagggcttggcccccatggggaagacacggatggaaggtggagcaaaggaaaatacatgaaattgagagtggcagctgcctgccaaaatctgttccgctgtaacagaactgaatttggaccccagccagtggctcacgcctgtaatcccagcactttggcaggccaaggtggaaggatcacttagagctaggagtttgagaccagcctgggcaatatgcaagacccctcactacaaaaataaaacatcaaaaacaaaaacaattagctgggcatgatggcacacacctgtgtccgagccacttgggaggctgggtgggaggatcggttgagcccaggagttcgaagctgcagggacctctgattgcaccactgcactccaggctgggtaacagaatgagccttatctcaaaaataaacaaactaataaaaagta

SEQ ID NO: 3 - protein, 578 aa, Homo sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGLLGPPRPQVLA

SEQ ID NO: 4 - DNA, 66 bp, Artificial Sequence
  oligonucleotide primer zc46250
ggtgtcctcgagaattcatataggccggccaccatgaaccggctccgggttgcacgcctcacgccg

SEQ ID NO: 5 - DNA, 43 bp, Artificial Sequence
  oligonucleotide primer zc47631
gcttgattgtgggccctctgggctccctgatcctattatctgg

SEQ ID NO: 6 - DNA, 22 bp, Artificial Sequence
  oligonucleotide primer zc24901
gagcccagagggcccacaatca

SEQ ID NO: 7 - DNA, 52 bp, Artificial Sequence
  oligonucleotide primer zc46896
gctgttttaaggcgcgcctctagatttatttacccggagtccgggagaagct

SEQ ID NO: 8 - DNA, 51 bp, Artificial Sequence
  oligonucleotide primer zc46759
tctggggtgggtacaaccccagagctgttttaaggcgcgcctctagattta

SEQ ID NO: 9 - DNA, 2226 bp, Artificial Sequence
  engineered fusion protein
atgaaccggctccgggttgcacgcctcacgccgttggagcttctgctgtcgctgatgtcgctgctgctcgggacgcggccccacggcagtccaggcccactgcagtgctacagcgtcggtcccctgggaatcctgaactgctcctgggaacctttgggcgacctggagactccacctgtgctgtatcaccagagtcagaaataccatcccaatagagtctgggaggtgaaggtgccttccaaacagagttgggtgaccattccccgggaacagttcaccatggctgacaaactcctcatctgggggacacaaaagggacggcctctgtggtcctctgtctctgtgaacctggagacccaaatgaagccagacacacctcagatcttctctcaagtggatatttctgaggaagcaaccctggaggccactgtgcagtgggcgccgcccgtgtggccaccgcagaaagctctcacctgtcagttccggtacaaggaatgccaggctgaagcatggacccggctggagccccagctgaagacagatgggctgactcctgttgagatgcagaacctggaacctggcacctgctaccaggtgtctggccgctgccaggtggagaacggatatccatggggcgagtggagttcgcccctgtccttccagacgccattcttagatcctgaagatgtgtgggtatcggggaccgtctgtgaaacttctggcaaacgggcagccctgcttgtctggaaggacccaagaccttgtgtgcaggtgacttacacagtctggtttggggctggagatattactacaactcaagaagaggtcccgtgctgcaagtcccctgtccctgcatggatggagtgggctgtggtctctcctggcaacagcaccagctgggtgcctcccaccaacctgtctctggtgtgcttggctccagaatctgccccctgtgacgtgggagtgagcagtgctgatgggagcccagggataaaggtgacctggaaacaagggaccaggaaaccattggagtatgtggtggactgggctcaagatggtgacagcctggacaagctcaactggacccgtctcccccctggaaacctcagcacattgttaccaggggagttcaaaggaggggtaccctatcgaattacagtgactgcagtatactctggaggattagctgctgcaccctcagtttggggattcagagaggagttagtaccccttgctgggccagcagtttggcgacttccagatgaccccccagggacacctgttgtagcctggggagaagtaccaagacaccagctcagaggccaggctactcactacaccttctgcatacagagcagaggcctctccactgtctgcaggaacgtgagcagtcaaacccagactgccactctgcccaaccttcactcgggttccttcaagctgtgggtgacggtgtccaccgttgcaggacagggcccacctggtcccgacctttcacttcacctaccagataataggatcagggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaataa

SEQ ID NO: 10 - protein, 741 aa, Artificial Sequence
  engineered fusion protein
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIREPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - DNA, 58 bp, Artificial Sequence
  oligonucleotide primer zc53405
aggtgtccagctcgagaattcatataggccggccaccatgcggggaggcaggggcgcc

SEQ ID NO: 12 - DNA, 63 bp, Artificial Sequence
  oligonucleotide primer zc51828
cggtgggcatgtgtgagttttgtctgaagatttgggctcggtgttatctggtagatgaagccg

SEQ ID NO: 13 - DNA, 63 bp, Artificial Sequence
  oligonucleotide primer zc51827
cctggtcccatcctccggcttcatctaccagataacaccgagcccaaatcttcagacaaaact

SEQ ID NO: 14 - DNA, 62 bp, Artificial Sequence
  oligonucleotide primer zc42508
ggggtgggtacaaccccagagctgttttaaggcgcgcctttatttacccggagacagggaga

SEQ ID NO: 15 - DNA, 2232 bp, Artificial Sequence
  engineered fusion protein
atgcggggaggcaggggcgcccctttctggctgtggccgctgcccaagctggcgctgctgcctctgttgtgggtgcttttccagcggacgcgtccccagggcagcgccgggccactgcagtgctacggagttggacccttgggcgacttgaactgctcgtgggagcctcttggggacctgggagccccctccgagttacacctccagagccaaaagtaccgttccaacaaaacccagactgtggcagtggcagccggacggagctgggtggccattcctcgggaacagctcaccatgtctgacaaactccttgtctggggcactaaggcaggccagcctctctggccccccgtcttcgtgaacctagaaacccaaatgaagccaaacgccccccggctgggccctgacgtggacttttccgaggatgaccccctggaggccactgtccattgggccccacctacatggccatctcataaagttctgatctgccagttccactaccgaagatgtcaggaggcggcctggaccctgctggaaccggagctgaagaccatacccctgacccctgttgagatccaagatttggagctagccactggctacaaagtgtatggccgctgccggatggagaaagaagaggatttgtggggcgagtggagccccattttgtccttccagacaccgccttctgctccaaaagatgtgtgggtatcagggaacctctgtgggacgcctggaggagaggaacctttgcttctatggaaggccccagggccctgtgtgcaggtgagctacaaagtctggttctgggttggaggtcgtgagctgagtccagaaggaattacctgctgctgctccctaattcccagtggggcggagtgggccagggtgtccgctgtcaacgccacaagctgggagcctctcaccaacctctctttggtctgcttggattcagcctctgccccccgtagcgtggcagtcagcagcatcgctgggagcacggagctactggtgacctggcaaccggggcctggggaaccactggagcatgtagtggactgggctcgagatggggaccccctggagaaactcaactgggtccggcttccccctgggaacctcagtgctctgttaccagggaatttcactgtcggggtcccctatcgaatcactgtgaccgcagtctctgcttcaggcttggcctctgcatcctccgtctgggggttcagggaggaattagcacccctagtggggccaacgctttggcgactccaagatgcccctccagggacccccgccatagcgtggggagaggtcccaaggcaccagcttcgaggccacctcacccactacaccttgtgtgcacagagtggaaccagcccctccgtctgcatgaatgtgagtggcaacacacagagtgtcaccctgcctgaccttccttggggtccctgtgagctgtgggtgacagcatctaccatcgctggacagggccctcctggtcccatcctccggcttcatctaccagataacaccgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 744 aa, Artificial Sequence [sequence omitted]

SEQ ID NO: 17 - protein, 813 aa, Mus musculus
MNRLERVALARLETPRLEGLLELELESLEMSLELELEGTRPRHGSPRGPRLEQCYSVGPRLEGILENCSWGLPRLEGDLEGLTPRPRVLEYHQSQKYHPRNRVWGLVKVPRSKQSWVTIPRRGLQFTMADKLELEIWGTQKGRPRLEWSSVSVASNLEGLTQMKPRDTPRQIFSQVASPISGLGLATLEGLATVQWAPRPRVWPRPRQKALETCQFRYKGLCQAGLAWTRLEGLPRQLEKTDGLETPRVGLMQNLEGLPRGTCYQVSGRCQVGLNGYPRWGGLWSSPRLESFQTPRFLEDPRGLDVWVSGTVCGLTSGKRAALELEVWKDPRRPRCVQVTYTVWFGAGDITTTQGLGLVPRCCKSPRVPRAWMGLWAVVSPRGNSTSWVPRPRTNLESLEVCLEAPRGLSAPRCDVGVSSADGSPRGIKVTWKQGTARGSPRLEGLYVVASPWAQDGDSLEDKLENWTRLEPRPRGNLESTLELEPRGGLFKGGVPRYRITVTAVYSGGLEAAAPRSVWGFRGLGLLEVPRLEAGPRAVWRLEPRDDPRPRGTPRVVALAWGGLVPRRHQLERGQATHYTFCIQSRGLESTVCRNVSSQTQTATLEPRNLEHSGSFKLEWVTVSTVALAGQGPRPRGPRDLESLEHLEPRDNRIRWKALEPRWFLESLEWGLELELEMGCGLESLEASTRCLEQARCLEHWRHKLELEPRQWIWGLRVPRDPRANSNSGQPRYIKGLVSLEPRQPRPRKDGPRILEGLVGLGLVGLLEQPRVVGLSPRKASAPRIYSGYGLKHFLEPRTPRGLGLLEGLELEV

SEQ ID NO: 18 - DNA, 18 bp, Artificial Sequence
  oligonucleotide primer zc10381
ccactacaccttgtgtgc

SEQ ID NO: 19 - DNA, 37 bp, Artificial Sequence
  oligonucleotide primer zc10390
tagtagcagatctgggctccctcagggtgttatctgg

SEQ ID NO: 20 - DNA, 20 bp, Artificial Sequence
  oligonucleotide primer zc10314
tcgtgattctctggtcggtg

SEQ ID NO: 21 - DNA, 20 bp, Artificial Sequence
  oligonucleotide primer zc10315
gtgattgctttggcggtgag

SEQ ID NO: 22 - DNA, 19 bp, Artificial Sequence
  oligonucleotide primer zc10382
gagttcaaaggaggggtac

SEQ ID NO: 23 - DNA, 40 bp, Artificial Sequence
  oligonucleotide primer zc10388
tagtagcagatctgggctccctgatcctattatctggtag

SEQ ID NO: 24 - DNA, 58 bp, Artificial Sequence; identical to SEQ ID NO: 11
  oligonucleotide primer zc53405

SEQ ID NO: 25 - DNA, 101 bp, Artificial Sequence
  oligonucleotide primer zc52311
tgggtacaaccccagagctgttttaaggcgcgcctctagattattttttagtgatggtgatggtgatgtccaccagatcccagggtgttatctggtagatg

SEQ ID NO: 26 - DNA, 1569 bp, Artificial Sequence
  Engineered fusion protein
atgcggggaggcaggggcgcccctttctggctgtggccgctgcccaagctggcgctgctgcctctgttgtgggtgcttttccagcggacgcgtccccagggcagcgccgggccactgcagtgctacggagttggacccttgggcgacttgaactgctcgtgggagcctcttggggacctgggagccccctccgagttacacctccagagccaaaagtaccgttccaacaaaacccagactgtggcagtggcagccggacggagctgggtggccattcctcgggaacagctcaccatgtctgacaaactccttgtctggggcactaaggcaggccagcctctctggccccccgtcttcgtgaacctagaaacccaaatgaagccaaacgccccccggctgggccctgacgtggacttttccgaggatgaccccctggaggccactgtccattgggccccacctacatggccatctcataaagttctgatctgccagttccactaccgaagatgtcaggaggcggcctggaccctgctggaaccggagctgaagaccatacccctgacccctgttgagatccaagatttggagctagccactggctacaaagtgtatggccgctgccggatggagaaagaagaggatttgtggggcgagtggagccccattttgtccttccagacaccgccttctgctccaaaagatgtgtgggtatcagggaacctctgtgggacgcctggaggagaggaacctttgcttctatggaaggccccagggccctgtgtgcaggtgagctacaaagtctggttctgggttggaggtcgtgagctgagtccagaaggaattacctgctgctgctccctaattcccagtggggcggagtgggccagggtgtccgctgtcaacgccacaagctgggagcctctcaccaacctctctttggtctgcttggattcagcctctgccccccgtagcgtggcagtcagcagcatcgctgggagcacggagctactggtgacctggcaaccggggcctggggaaccactggagcatgtagtggactgggctcgagatggggaccccctggagaaactcaactgggtccggcttccccctgggaacctcagtgctctgttaccagggaatttcactgtcggggtcccctatcgaatcactgtgaccgcagtctctgcttcaggcttggcctctgcatcctccgtctgggggttcagggaggaattagcacccctagtggggccaacgctttggcgactccaagatgcccctccagggacccccgccatagcgtggggagaggtcccaaggcaccagcttcgaggccacctcacccactacaccttgtgtgcacagagtggaaccagcccctccgtctgcatgaatgtgagtggcaacacacagagtgtcaccctgcctgaccttccttggggtccctgtgagctgtgggtgacagcatctaccatcgctggacagggccctcctggtcccatcctccggcttcatctaccagataacaccctgggatctggtggacatcaccatcaccatcac

SEQ ID NO: 27 - protein, 692 aa, Artificial Sequence
  Engineered fusion protein
MRGGRGAPRFWLEWPRLEPRKLEALELEPRLELEWVLEFQRTRPRQGSAGPRLEQCYGVGPRLEGDLENCSWGLPRLEGDLEGAPRSGLLEHLEQSQKYRSNKTQTVALAVALAAGRSWVALAIPRRGLQLETMSDKLELEVWGTKAGQPRLEWPRPRVFVASNLEGLTQMKPRNAPRRLEGPRDVASPFSGLDDPRLEGLATVHWAPRPRTWPRSHKVLEICQFHYRRCQGLAAWTLELEGLPRGLLEKTIPRLETPRVGLIQDLEGLLEATGYKVYGRCRMGLKGLGLDLEWGGLWSPRILESFQTPRPRSAPRKDVWVSGNLECGTPRGGGLGLPRLELELEWKAPRGPRCVQVSYKVWFWVGGRGLLESPRGLGITCCCSLEIPRSGAGLWARVSAVASNATSWGLPRLETNLESLEVCLEDSASAPRRSVALAVSSIAGSTGLLELEVTWQPRGPRGGLPRLEGLHVVASPWARDGDPRLEGLKLENWVARGLEPRPRGNLESALELEPRGNFTVGVPRYRITVTAVSASGLEASASSVWGFRGLGLLEAPRLEVGPRTLEWRLEQDAPRPRGTPRAIAWGGLVPRRHQLERGHLETHYTLECAQSGTSPRSVCMNVSGNTQSVTLEPRDLEPRWGPRCGLLEWVTASTIAGQGPRPRGPRILERLEHLEPRDNTLEGSGGHHHHHH

(19) United States
(12) Patent Application Publication
(10) Pub. No.: US 2010/0093076 A1
(43) Pub. Date: Apr. 15, 2010

(54) SOLUBLE IL-27 RECEPTOR

(76) Inventor: Margaret D. Moore, Seattle, WA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/438,905

(22) PCT Filed: Aug. 27, 2007
(86) PCT No.: PCT/US07/76897

§ 371 Date: Dec. 14, 2009

Related U.S. Application Data
(60) Provisional application No. 60/823,558, filed on Aug. 25, 2006.
(60) Provisional application No. 60/871,080, filed on Dec. 20, 2006.

(51) Int. Cl. C07K 16/00; C07H 21/04; C12N 15/63

(52) U.S. Cl. 435/320.1; 530/391.1; 536/23.53

(57) ABSTRACT

Polypeptides and proteins comprising an IL-27RA cytokine binding domain operably linked to an immunoglobulin Fc fragment are disclosed. The Fc fragment is a modified Fc fragment wherein amino acid residues at EU index postions 234, 235, and 237 have been substituted to reduce binding to FcγRI and amino acid residues at EU index positions 330 and 331 have been substituted to reduce complement fixation. The proteins can be used medicinally for modulating immune responses, such as within methods of treating autoimmune diseases.

### BACKGROUND

Interleukin-27 (IL-27) is a cytokine that has been reported to promote the development of Th1-type CD4 T-cell responses and inhibit the development of Th2 responses, to stimulate the production of inflammatory cytokines by non-T-cells, including cytokines necessary to sustain a Th1 response (e.g. IL-12 and IL-18), and to suppress the development of IL-17-producing T cells. See, Brombacher et al., Trends Immunol 24(4): 207-212, 2003; Hunter, Nature Reviews Immunology 5:521-531, 2005; and Stumhofer et al., Nat. Immunol. 7:937-945, 2006. IL-27 is a heterodimer of the polypeptide subunits Epstein-Barr virus-induced gene 3 (EBI3) and IL-27 p28 (Pflanz et al., Immunity 16:779-790, 2002). IL-27 binds to a heterodimeric cell-surface receptor composed of the subunits gp130 and IL-27RA. The latter is also known as WSX-1 (Sprecher et al., Biochem. Biophys. Res. Comm., 246:82-98, 1998), zcytorl (Baumgartner et al., U.S. Pat. No. 5,792,850), and TCCR (Chen et al., Nature 407:916-920, 2000). Mice deficient in IL-27RA have been reported to show higher levels of protective immunity against Mycobacterium tuberculosis infection than wild-type mice, but to develop an ultimately fatal, increased chronic inflammatory response (Hölscher et al., J. Immunol. 174:3534-3544, 2005). Hunter et al., US 2004/0185049 A1 disclose that agonist ligands of IL-27RA can be used to treat immune hyperreactivity, including Th1-mediated and Th2-mediated diseases. IL-27 antagonists have been proposed for treatment of autoimmune diseases, including multiple sclerosis, rheumatoid arthritis, inflammatory bowel disease, and Crohn's disease; and for treatment of leukemia and lymphoma. See for example, Baumgartner et al., ibid.; Goldberg et al., J. Immunol. 173:1171-1178, 2004; De Sauvage et al., WO 01/29070. Experimental evidence also suggest that neutralization of IL-27 may be beneficial in the treatment of sepsis (Wirtz et al., J. Exp. Med. 203:1875-1881, 2006).

There is a need in the art for therapeutic agents that antagonize IL-27 activity and have favorable pharmacokinetic properties. Thus, provided herein are IL27 antagonists, as well as other, related advantages.

### SUMMARY

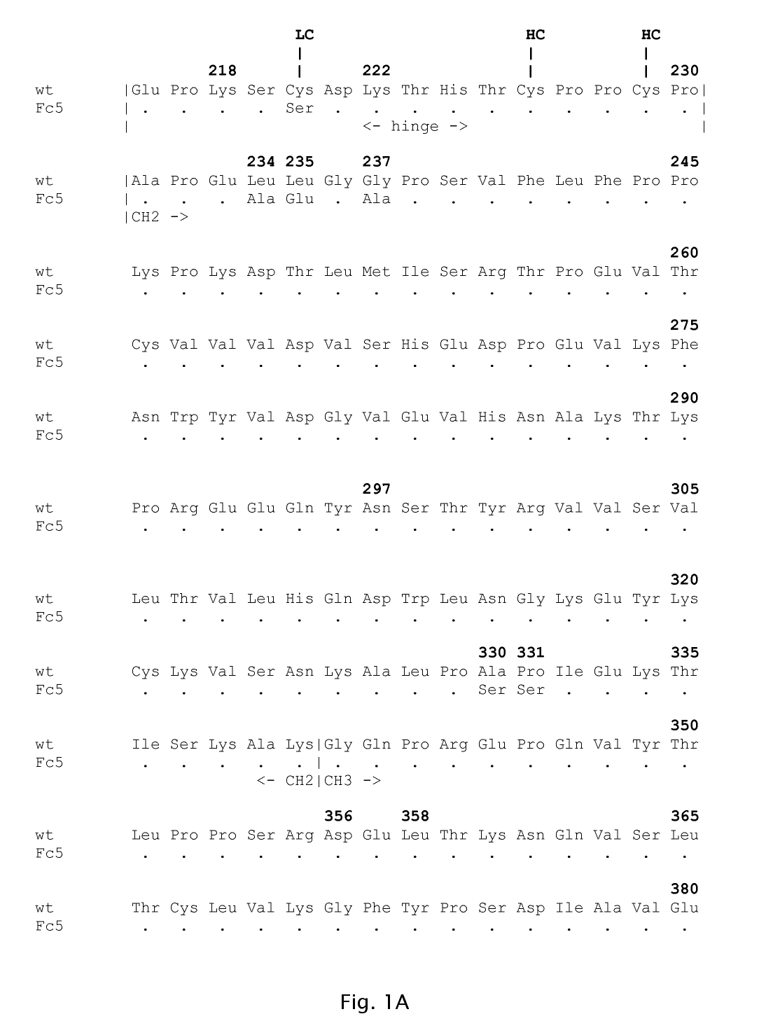

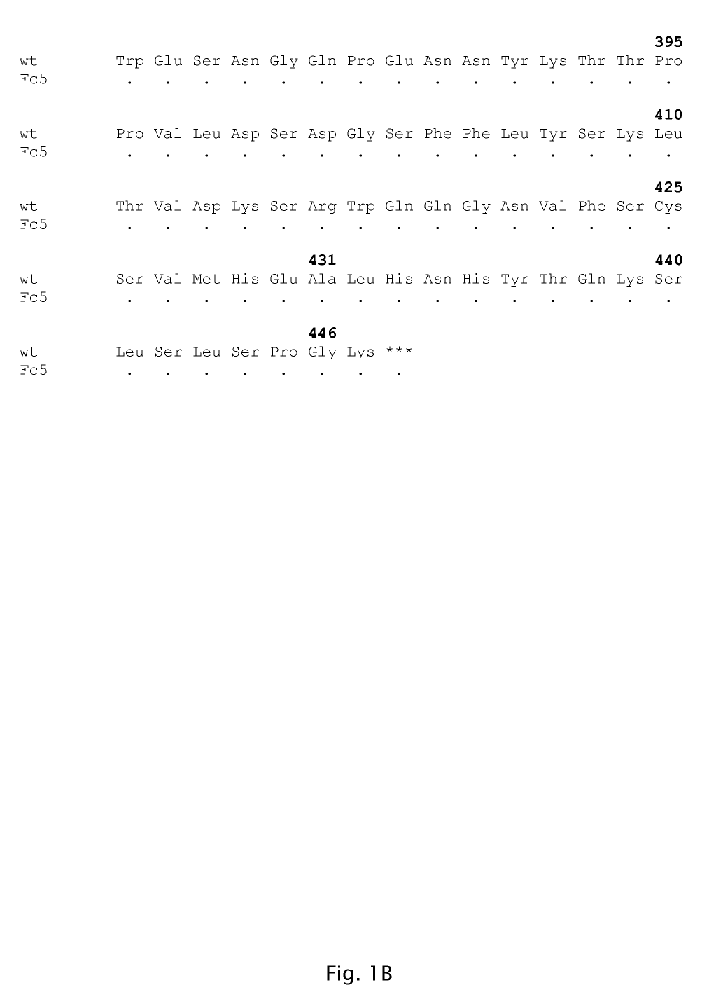

Disclosed herein are polypeptides comprising, from amino terminus to carboxyl terminus, a cytokine binding domain operably linked to an immunoglobulin Fc fragment, wherein the cytokine binding domain is substantially similar to the cytokine binding domain of an IL27RA polypeptide and wherein the Fc fragment is a modified Fc fragment with substituted amino acid residues at EU index positions 234, 235, and 237 to reduce binding to Fc.gamma.RI, and substituted amino acid residues at EU index positions 330 and 331 to reduce complement fixation. Within certain embodiments, the IL-27RA cytokine binding domain is a human IL27RA cytokine binding domain. Within certain embodiments, the Fc fragment is a human Fc fragment. Within further embodiments, the Fc fragment is further modified by substitution of a cysteine residue at EU index position 220. Within other embodiments, the Fc fragment is an Fc5 fragment as shown in FIGS. 1A-1B. Within certain embodiments, the IL27RA-Fc fragment polypeptide consists of amino acid residues 33 to 744 of SEQ ID NO:16, and the polypeptide is optionally glycosylated.

Also provided are polypeptides consisting essentially of, from amino terminus to carboxyl terminus, an IL-27RA extracellular domain operably linked to an immunoglobulin Fc fragment, wherein the Fc fragment is a modified Fc fragment wherein amino acid residues at EU index positions 234, 235, and 237 have been substituted to reduce binding to Fc.gamma.RI and amino acid residues at EU index positions 330 and 331 have been substituted to reduce complement fixation. Within certain embodiments, the IL-27RA extracellular domain is a human IL27RA extracellular domain. Within additional embodiments, the Fc fragment is a human Fc fragment. Within further embodiments, the Fc fragment is further modified by substitution of a cysteine residue at EU index position 220. Within other embodiments, the Fc fragment is an Fc5 fragment as shown in FIGS. 1A-1B.

Further provide are dimeric proteins consisting of two polypeptides joined by a disulfide bond, each of the polypeptides comprising, from amino terminus to carboxyl terminus, an IL-27RA extracellular domain operably linked to an immunoglobulin Fc fragment, wherein the Fc fragment is a modified Fc fragment wherein amino acid residues at EU index positions 234, 235, and 237 have been substituted to reduce binding to Fc.gamma.RI and amino acid residues at EU index positions 330 and 331 have been substituted to reduce complement fixation, wherein the protein binds IL-27. Within certain embodiments, the IL-27RA extracellular domain is a human IL27RA extracellular domain. Within other embodiments, the extracellular domain consists of residues 33 to 512 of SEQ ID NO:3 and is optionally glycosylated. Within further embodiments, the Fc fragment is a human Fc fragment. Within additional embodiments, the Fc fragment is further modified by substitution of a cysteine residue at EU index position 220. Within still other embodiments, the Fc fragment is an Fc5 fragment as shown in FIGS. 1A-1B. Within certain embodiments, each of the IL27RA-Fc fragment polypeptides consists of amino acid residues 33 to 744 of SEQ ID NO:16, and each of the polypeptides is optionally glycosylated. Within other embodiments, one of the IL27RA-Fc fragment polypeptides consists of amino acid residues 33 to 744 of SEQ ID NO:16, and independently each of the polypeptides is optionally glycosylated.

Also provided are methods of modulating an immune response in a patient, comprising administering to a patient in need thereof a composition comprising a polypeptide or protein as disclosed above in combination with a pharmaceutically acceptable vehicle. Within certain embodiments, the polypeptide or protein is used within a method of treating an autoimmune disease in a patient, comprising administering to a patient in need thereof a composition comprising the polypeptide or protein in combination with a pharmaceutically acceptable vehicle. Within other embodiments, the polypeptide or protein is used within a method of suppressing a Th1 immune response in a patient, comprising administering to a patient in need thereof a composition comprising the polypeptide or protein in combination with a pharmaceutically acceptable vehicle.

These and other aspects will become evident upon reference to the following detailed description and the attached drawings.

### BRIEF DESCRIPTION OF THE FIGURES

The drawing (FIGS. 1A-1B) illustrates the amino acid sequences of certain immunoglobulin Fc polypeptides (SEQ ID NO:1). Amino acid sequence numbers are based on the EU index (Kabat et al., Sequences of Proteins of Immunological Interest, US Department of Health and Human Services, NIH, Bethesda, 1991). The illustrated sequences include a wild-type human sequence (“wt”) and a variant sequence designated Fc5. The Cys residues normally involved in disulfide bonding to the light chain constant region (LC) and heavy chain constant region (HC) are indicated. A “.” indicates identity to wild-type at that position. A “***” notation indicates the stop codon. Boundaries of the hinge, C_{H}2, and C_{H}3 domains are shown.

### DESCRIPTION

An immunoglobulin “Fc fragment” (or Fc domain) is the portion of an antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, C_{H}2, and C_{H}3 domains. However, the term has more recently been applied to a single chain consisting of C_{H}3, C_{H}2, and at least a portion of the hinge sufficient to form a disulfide-linked dimer with a second such chain. For a complete review of immunoglobulin structure and function see Putnam, The Plasma Proteins, Vol V, Academic Press, Inc., 49-140, 1987; and Padlan, Mol. Immunol. 31:169-217, 1994. As used herein, the term Fc includes variants of naturally occurring sequences.

“Operably linked”, when referring to polypeptides, indicates that the polypeptides are connected by a peptide bond or an additional polypeptide and are arranged so that they function in concert for their intended purposes.

A “polypeptide” is a polymer of amino acid residues joined by peptide bonds, whether produced naturally or synthetically. Polypeptides of less than about 10 amino acid residues are commonly referred to as “peptides”. A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins and polypeptides are defined herein in terms of their amino acid backbone structures; while substituents such as carbohydrate groups are generally not specified, but may be present nonetheless.

Provided herein are proteins that antagonize the activity of interleukin-27. The proteins comprise a soluble IL-27RA polypeptide region operably linked to a modified immunoglobulin Fc polypeptide region, thereby providing unexpectedly favorable pharmacokinetic properties. The disclosed proteins can be used as therapeutic agents, such as in the treatment of autoimmune diseases.

A representative human IL-27RA protein is shown in SEQ ID NO:3 This protein has been disclosed in U.S. Pat. No. 5,792,850, wherein it is referred to as “Zcytor1.” Features of the protein an extracellular domain, a transmembrane domain and a intracellular signaling domain. With reference to the amino acid chain in SEQ ID NO:3, the approximate boundaries of the extracellular domain are from about residue 1 to about residue 514, the transmembrane domain is from about residue 515 to about residue 540 and the intracellular signaling domain is from about residue 541 to about residue 578. The extracellular domain further comprises a WSXWS motif at residues 217-221 of SEQ ID NO:3, a cytokine-binding domain of approximately 200 amino acid residues (residues 33 to 235 of SEQ ID NO:3), and three fibronectin type III domains (residues 236 to 514 of SEQ ID NO:3). The cytokine-binding domain and fibronectin type III domains collectively have approximate boundaries from about residue 33 to about residue 514 of SEQ ID NO:3. Those skilled in the art will recognize that the stated domain boundaries are approximate and are based on alignments with known proteins and predictions of protein folding; functional domain boundaries may vary by ±5 residues from the stated positions. In addition to these domains, conserved receptor features in the receptor protein include (with reference to SEQ ID NO:3) a Cys-X-Trp domain at residues 52-54, a Cys residue at position 41, a Trp residue at position 151, and an Arg residue at position 207.

The proteins provided herein comprise an extracellular binding domain of IL-27RA (or “Zcytor1 fragment”) joined to a multimerizing protein as generally disclosed in Sledziewski et al., U.S. Pat. Nos. 5,155,027 and 5,567,584. See also, Baumgartner et al., U.S. Pat. No. 5,792,850. Within the present disclosure, the multimerizing protein is an immunoglobulin Fc fragment. It is known in the art that Ig constant region domains may be fused to other polypeptides to increase their the circulatory half-life or to add antibody-dependent effector functions. Fusion to an Fc fragment may also improve the production characteristics of a protein of interest. Immunoglobulin Fc fusion proteins are typically secreted from recombinant host cells as multimeric molecules wherein the Fc portions are disulfide bonded to each other, and the two non-Ig polypeptides (e.g., receptor fragments) are arrayed in close proximity to each other. As disclosed in more detail below, however, the inventors have found that an IL-27RA extracellular domain polypeptide joined to a wild-type IgG Fc fragment was rapidly cleared from the circulation of experimental animals. Circulation half-life was markedly improved when a variant Fc fragment (termed “Fc5”) was contained in the fusion protein. As shown in FIGS. 1A-1B, the Fc5 variant includes amino acid substitutions at EU index positions 234, 235, and 237 to reduce binding to the high affinity Fc gamma receptor (Fc.gamma.RI), and at EU index positions 330 and 331 to reduce complement fixation. See, Duncan et al., Nature 332:563-564, 1988; Winter et al., U.S. Pat. No. 5,624,821; Tao et al., J. Exp. Med. 178:661-667, 1993; and Canfield and Morrison, J. Exp. Med. 173:1483-1491, 1991. As also shown in FIGS. 1A-1B, the Cys residue within the hinge region that is ordinarily disulfide-bonded to the light chain (EU index position 220) was replaced with a serine residue to eliminate an unpaired cysteine in the dimeric protein.

Provided herein are polypeptides comprising, from amino terminus to carboxy terminus, a Zcytor1 fragment operably linked to an immunoglobulin Fc fragment (or “IL-27RA/Fc fusions”). The Zcytor1 fragment preferably has at least 80% amino acid sequence identity with the amino acid structure of the extracellular domain of SEQ ID NO: 3, though said fragment may have at least 80% amino acid sequence identity with amino acid residue 1 to amino acid residue 578 of SEQ ID NO:3. Thus, said Zcytor1 fragment may comprise one or more of the extracellular domain, the transmembrane domain, the intracellular signaling domain, the cytokine binding domain, a fibronectin domain, a plurality of fibronectin domains and a plurality of cytokine binding domains. In one embodiment, said Zcytor1 fragment has an amino acid sequence that is at least 80% identical to residue 1 to about residue 514 of SEQ ID NO:3. In another embodiment, said Zcytor1 fragment has an amino acid sequence that is at least 80% identical to residues 33 to 514 of SEQ ID NO:3. In another embodiment, said Zcytor1 fragment has an amino acid sequence that is at least 80% identical to residues 33 to 235 of SEQ ID NO:3. In a still further embodiment, said Zcytor1 fragment comprises one or more of said conserved residues, with reference to SEQ ID NO:3: a Cys-X-Trp domain at residues 52-54, a Cys residue at position 41, a Trp residue at position 151, and an Arg residue at position 207.

As is used herein, the term “at least 80% identity” means that an amino acid sequence shares 80%-100% identity with a reference sequence. This range of identity is inclusive of all whole (e.g., 85%, 87%, 93%, 98%) or partial numbers (e.g., 87.27%, 92.83%, 98.11%- to two significant figures) embraced within the recited range numbers, therefore forming a part of this description. For example, an amino acid sequence with 200 residues that share 85% identity with a reference sequence would have 170 identical residues and 30 non-identical residues. Similarly, the amino acid sequence may have 200 residues that are identical to a reference sequence that is 235 residues in length, thus the amino acid sequence will be 85.11% identical to the larger reference sequence. This scenario is more typical when an amino acid sequence is a portion of a domain on the reference sequence. Amino acid sequences may additionally vary in percent identity from a reference sequence by way of both size differences and residue mis-matches. Those ordinarily skilled in the are will readily calculate percent identity between an amino acid and a reference sequence.

The proteins provided herein can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms). Cells derived from higher eukaryotic organisms, particularly cultured mammalian cells, are preferred for production of the instantly disclosed proteins. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, Green and Wiley and Sons, NY, 1993.

In general, a DNA sequence encoding an IL-27RA/Fc fusion polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a protein into the secretory pathway of a host cell, a secretory signal sequence (also known as a leader sequence, prepro sequence or pre sequence) is provided in the expression vector. The secretory signal sequence may be that belonging to the IL-27RA polypeptide itself, or it may be derived from another secreted protein (e.g., t-PA; see, U.S. Pat. No. 5,641,655) or synthesized de novo. The secretory signal sequence is operably linked to the DNA sequence encoding the fusion protein, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Both during and after construction of an expression vector, the vector is typically propagated in a prokaryotic or lower eukaryotic host cell. Prokaryotic host cells useful in this regard include E. coli and other species known in the art. Suitable strains of E. coli include, but are not limited to, BL21(DE3), BL21(DE3)pLysS, BL21(DE3)pLysE, DH1, DH4I, DH5, DH5I, DH5IF′, DH51MCR, DH10B, DH10B/p3, DH11S, C600, HB101, JM101, JM105, JM109, JM110, K38, RR1, Y1088, Y1089, CSH18, ER1451, and ER1647 (see, for example, Brown (ed.), Molecular Biology Labfax, Academic Press, 1991). Standard techniques for propagating vectors in prokaryotic hosts are also well-known to those of skill in the art (see, for example, Ausubel et al. (eds.), Short Protocols in Molecular Biology, 3^{rd} Edition, John Wiley & Sons, 1995 and Wu et al., Methods in Gene Biotechnology, CRC Press, Inc., 1997). Yeast species of particular interest in this regard include Saccharomyces cerevisiae, Pichia pastoris, and Pichia methanolica. Methods for transforming and culturing bacterial and yeast cells are well known in the art and are disclosed in more detail below.

Expression of IL-27RA/Fc fusion proteins via a host cell secretory pathway is expected to result in the production of multimeric (e.g., dimeric) proteins. If the fusion protein is to be produced as a dimer without associated immunoglobulin light chains, host cells that do not produce endogenous immunoglobulins are preferred as hosts, and the Fc portion of the fusion will preferably be modified to eliminate any unpaired cysteine residues. Multimers may also be assembled in vitro upon incubation of component proteins under suitable conditions. In general, in vitro assembly will include incubating the polypeptide mixture under denaturing and reducing conditions followed by refolding and reoxidation of the polypeptides to form dimers. Again, assembly of properly folded dimers is facilitated by elimination of unpaired cysteine residues. Recovery and assembly of proteins expressed in bacterial cells is disclosed below.

Cultured mammalian cells are suitable hosts for production of IL-27 antagonists. Methods for introducing exogenous DNA into mammalian host cells include, but are not limited to, calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., 1993, ibid.), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB11 (Hyclone, Logan, Utah); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Va. Strong transcription promoters can be used, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” Exemplary selectable markers include a gene encoding resistance to the antibiotic neomycin, which allows selection to be carried out in the presence of a neomycin-type drug, such as G-418 or the like; the gpt gene for xanthine-guanine phosphoribosyl transferase, which permits host cell growth in the presence of mycophenolic acid/xanthine; and markers that provide resistance to zeocin, bleomycin, blastocidin, and hygromycin (see, e.g., Gatignol et al., Mol. Gen. Genet. 207:342, 1987; Drocourt et al., Nucl. Acids Res. 18:4009, 1990). Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

Insect cells can be infected with recombinant baculovirus, commonly derived from Autographa californica nuclear polyhedrosis virus (AcNPV). See, King and Possee, The Baculovirus Expression System: A Laboratory Guide, Chapman & Hall, London; O'Reilly et al., Baculovirus Expression Vectors: A Laboratory Manual, Oxford University Press., New York, 1994; and Richardson, Ed., Baculovirus Expression Protocols. Methods in Molecular Biology, Humana Press, Totowa, N.J., 1995. Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, Md.). The transfer vector (e.g., PFASTBAC1; Life Technologies) contains a Tn7 transposon to move the DNA encoding the polypeptide of interest into a baculovirus genome maintained in E. coli as a large plasmid called a “bacmid.” See, Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing an IL-27RA/Fc fusion-encoding sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the fusion protein is subsequently produced. Recombinant viral stocks are made by methods commonly used in the art.

For protein production, the recombinant virus is used to infect host cells, typically a cell line derived from the fall armyworm, Spodoptera frugiperda (e.g., Sf9 or Sf21 cells) or Trichoplusia ni (e.g., HIGH FIVE cells; Invitrogen, Carlsbad, Calif.). See, in general, Glick and Pasternak, Molecular Biotechnology, Principles & Applications of Recombinant DNA, ASM Press, Washington, D.C., 1994. See also, U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (e.g., King and Possee, ibid.; O'Reilly et al., ibid.; Richardson, ibid.).

Fungal cells, including yeast cells, can also be used herein. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936; and 4,661,454. Transformation systems for other yeasts, including Hansenula polymorphs, Schizosaccharomyces pombe, Kluyveromyces lactis, Kluyveromyces fragilis, Ustilago maydis, Pichia pastoris, Pichia methanolica, Pichia guillermondii, and Candida maltosa are known in the art. See, for example, Gleeson et al., J. Gen. Microbiol. 132:3459-3465, 1986; Cregg, U.S. Pat. No. 4,882,279; and Raymond et al., Yeast 14:11-23, 1998. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus and other genera are also useful host cells. Techniques for transforming these hosts and expressing foreign DNA sequences cloned therein are well known in the art (see, e.g., Sambrook et al., ibid.). When expressing an IL-27RA/Fc fusion in bacteria such as E. coli, the protein may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured protein can then be refolded and dimerized by diluting the denaturant, such as by dialysis against a solution of urea and a combination of reduced and oxidized glutathione, followed by dialysis against a buffered saline solution. In the alternative, the protein may be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

The proteins disclosed herein can be purified by conventional purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Because the disclosed IL-27RA/Fc fusion polypeptides comprise an immunoglobulin heavy chain polypeptide region, they can be purified by affinity chromatography on immobilized protein A. Additional purification steps, such as gel filtration or size exclusion chromatography, can be used to obtain the desired level of purity or to provide for desalting, buffer exchange, and the like.

The disclosed proteins may be used in medicine to modulate an immune response in a patient. In particular, the proteins may be used to suppress Th1 immune responses, to promote development of Th2 immune responses, and/or to increase the expansion of regulatory T cells (Treg) relative to other T cell subsets. IL-27 has been found to inhibit the development and survival of CD4⁺ FoxP3⁺ Treg. Treg are important for maintenance of self-tolerance and restraining T cell responses. Therefore, neutralization of IL-27 may inhibit the development of autoimmune responses and graft-versus-host disease. Thus, the instant proteins may be used in the treatment of autoimmune diseases (including, but not limited to, multiple sclerosis, rheumatoid arthritis, inflammatory bowel disease, Crohn's disease, and sarcoidosis), graft-versus-host disease, aplastic anemia, sepsis, leukemia, and lymphoma.

For pharmaceutical use, the IL-27RA/fc fusion polypeptides disclosed herein are formulated for topical or parenteral delivery, particularly intravenous, intramuscular, or subcutaneous, delivery according to conventional methods. In general, pharmaceutical formulations will include an IL-27 antagonist in combination with a pharmaceutically acceptable vehicle, such as saline, buffered saline, 5% dextrose in water, or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. A “therapeutically effective amount” of an IL-27RA/Fc fusion polypeptide is that amount that produces a statistically significant effect, such as a statistically significant reduction in disease progression or a statistically significant improvement in organ function. The exact dose will be determined by the clinician according to accepted standards, taking into account the nature and severity of the condition to be treated, patient traits, etc. Determination of dose is within the level of ordinary skill in the art. The therapeutic formulations will generally be administered over the period required to achieve a beneficial effect, commonly from several weeks up to several months and, in treatment of chronic conditions, for a year or more with periodic evaluations (e.g., at 3-month intervals) for clinical response. Dosing is daily or intermittently (e.g., one, two, three, or more times per week) over the period of treatment. Intravenous administration will be by bolus injection or infusion over a typical period of one to several hours. Sustained release formulations can also be employed. An IL-27 antagonist may also be delivered by aerosolization according to methods known in the art. See, for example, Wang et al., U.S. Pat. No. 5,011,678; Gonda et al., U.S. Pat. No. 5,743,250; and Lloyd et al., U.S. Pat. No. 5,960,792. The proteins described herein will commonly be administered at doses of 0.01 to 10 mg/kg of patient body weight, generally from 0.1 to 10 mg/kg, more often 1.0 to 10 mg/kg in multiple administrations (typically by injection or infusion). Larger loading doses may be followed by smaller maintenance doses over the course of treatment.

The following examples are provided as illustration and not for limitation.

### EXAMPLES

### Example 1

A DNA construct encoding a mouse IL27RA-Fc fusion polypeptide comprising the extracellular domain of mouse IL27RA and a wild type BALB/c mouse.gamma.2a constant region Fc tag (designated “IL27RAm(mFc1)”) was constructed via a 3-step PCR and homologous recombination using a DNA fragment encoding the extracellular domain of mouse IL27RA and the expression vector pZMP40. Plasmid pZMP40 is a mammalian expression vector containing an expression cassette comprising the chimeric CMV enhancer/MPSV promoter, a BglII site for linearization prior to yeast recombination, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae. pZMP40 is a derivative of plasmid pZMP21, which is described in US patent application publication No. 2003/0232414 Al and has been deposited at the American Type Culture Collection, 10801 University Boulevard, Manassas, Va. 20110-2209, designated No. PTA-5266.

A PCR fragment encoding IL27RAm(mFc1) was constructed to contain a 5′ overlap with the pZMP40 vector sequence in the 5′ non-translated region, the IL27RA extracellular domain coding region, the C-terminal mFc1 tag coding sequence, and a 3′ overlap with the pZMP40 vector in the poliovirus internal ribosome entry site region. The first PCR amplification reaction used the 5′ oligonucleotide primer zc46250 (SEQ ID NO:4), the 3′ oligonucleotide primer zc47631 (SEQ ID NO:5), and a previously generated plasmid containing mouse IL27RA cDNA as the template. A second PCR fragment was generated using the 5′ oligonucleotide primer zc24901 (SEQ ID NO:6), the 3′ oligonucleotide primer zc46896 (SEQ ID NO:7) and a previously generated plasmid containing mouse Ig gamma2a Fc cDNA (designated “mFc1”) as the template. The PCR amplification reaction conditions were as follows: One cycle of 95° C. for 5 minutes; then 35 cycles of 95° C. for 30 seconds, 55° C. for 30 seconds, and 68° C. for 2 minutes; then one cycle of 68° C. for 10 minutes; followed by a 4° C. hold. The PCR reaction mixtures were run on a 1.2% agarose gel, and the DNA fragments corresponding to the expected size were extracted from the gel using a commercially available gel extraction kit (QIAQUICK Gel Extraction Kit; QIAGEN Inc., Valencia, Calif.).

The two fragments were then joined and amplified using the 5′ oligonucleotide primer zc46250 (SEQ ID NO:4) and the 3′ oligonucleotide primer zc46759 (SEQ ID NO:8) under the following PCR conditions: one cycle of 95° C. for 3 minutes; then 35 cycles of 95° C. for 30 seconds and 72° C. for 2 minutes; then one cycle of 72° C. for 7 minutes; followed by a 4° C. hold. The final PCR product was cloned using a commercially available kit (TOPO TA CLONING Kit; Invitrogen, Carlsbad, Calif.) according to the manufacturer's directions. Two μl of the cloning reaction mixture was used to transform chemically competent E. coli cells (ONE SHOT DH10B-T1; Invitrogen), which were plated onto LB AMP plates (LB broth (Lennox), 1.8% BACTO Agar (DIFCO), 100 mg/L Ampicillin) overnight. Colonies were sequenced and found to have deletions within the IL27RA coding region. This discrepancy was resolved by performing a double digest with KpnI and SpeI on two clones and ligating the two correct fragments using a commercially available DNA ligation kit (FAST-LINK; EPICENTRE Biotechnologies, Madison, Wis.) according to the manufacturer's protocol. A resulting colony that contained the corrected insert sequence was grown up in LB AMP broth, and the plasmid was purified with a commercially available kit (QIAPREP Spin Miniprep kit; QIAGEN Inc.). The plasmid clone was then digested with EcoRI, and the IL27RAm(mFc1) insert was excised and purified using a commercially available gel extraction kit (QIAQUICK Gel Extraction Kit).

The plasmid pZMP40 was digested with B gill prior to recombination in yeast with the purified IL27RAm(mFc1) fragment. One hundred μL of competent yeast (S. cerevisiae) cells were combined with 10 μL (1.micro.g) of the IL27RAm(mFc1) insert DNA and 100 ng of BglII-digested pZMP40 vector, and the mixture was transferred to a 0.2-cm electroporation cuvette. The yeast/DNA mixture was electropulsed using power supply (BIORAD Laboratories, Hercules, Calif.) settings of 0.75 kV (5 kV/cm), ∞ ohms, and 25 μF. Six hundred μL of 1.2 M sorbitol was added to the cuvette, and the yeast was plated in 300-μL aliquots onto two URA-D plates and incubated at 30° C. After about 72 hours, the Ura⁺ yeast transformants from a single plate were resuspended in 1 ml H₂O and spun briefly to pellet the yeast cells. The cell pellet was resuspended in 500 μL of lysis buffer (2% t-octylphenoxypolyethoxyethanol (TRITON X-100), 1% SDS, 100 mM NaCl, 10 mM Tris, pH 8.0, 1 mM EDTA). The 500 μL of the lysis mixture was added to a microcentrifuge tube containing 300 μL acid-washed glass beads and 200 μL phenol-chloroform, vortexed for 2 minutes, and spun for 5 minutes in a microcentrifuge at maximum speed. Three hundred μL of the aqueous phase was transferred to a fresh tube, and the DNA was precipitated with 600 μL ethanol, followed by centrifugation for 10 minutes at maximum speed. The tube was decanted, and the DNA pellet was resuspended in 10 μL deionized H₂O.

Transformation of electrocompetent E. coli host cells (DH10B) was performed using one μL of the yeast DNA preparation and 25 μl of E. coli cells. The cells were electropulsed at 2.5 kV, 25 μF, and 200 ohms. Following electroporation, 1 ml SOC (2% BACTO Tryptone (DIFCO, Detroit, Mich.), 0.5% yeast extract (DIFCO), 10 mM NaCl, 2.5 mM KCl, 10 mM MgCl₂, 10 mM MgSO₄, 20 mM glucose) was added, and the cells were plated in 100-μL and 500-μL aliquots on two LB AMP plates. The inserts of three DNA clones for the construct were subjected to sequence analysis, and one clone containing the correct sequence was selected. Large-scale plasmid DNA was isolated using a commercially available kit (QIAGEN ENDOFREE Plasmid Mega Kit; QIAGEN Inc.) according to the manufacturer's instructions. The sequence of the insert DNA is shown in SEQ ID NO:9.

For transfection into CHO cells, 600 μg of the IL27RAm(mFc1)/pZMP40 expression plasmid was digested with 600 units of BstB1 at 37° C. for three hours, purified via phenol-chloroform extraction, and aliquoted to three microcentrifuge tubes. 0.1 volume 3M NaOAC, pH 5.2, and 2.2 volumes ethanol were added to each tube, and the tubes were stored on ice until transfection. The DNA was then spun down in a microfuge for 10 minutes at 14,000 RPM, and the supernatant was decanted off each pellet. The pellets were washed with 70% ethanol, decanted, and allowed to air dry for 15 minutes, then resuspended in 200 μL each of CHO cell culture medium in a sterile environment and allowed to incubate at 37° C. until the DNA pellets dissolved. Three tubes of approximately 1×10⁷ CHO DXB11 cells from log-phase culture were pelleted and resuspended in 600 μL warm medium. The DNA/cell mixtures were combined and placed in three 0.4-cm gap cuvettes and electroporated at 950 μF, high capacitance, 300 V. The contents of each cuvette was removed and diluted to 20 mL with CHO cell culture medium and placed in a 125-mL shake flask. The flasks were placed in a 37° C., 5% CO₂ incubator on a shaker platform set at 120 RPM. After approximately 48 hours, the contents of the three flasks were pooled and subjected to nutrient selection and step amplification to 200 nM methotrexate (MTX), and then to 1 μM MTX. Tagged protein expression was confirmed by Western blot, and the CHO cell pool was scaled-up for harvests for protein purification.

### Example 2

An expression plasmid encoding a human IL27RA-Fc5 fusion protein was constructed via homologous recombination in yeast. DNA fragments encoding the extracellular domain and secretion leader peptide of human IL27RA (amino acids 1 to 512 of SEQ ID NO:3) and Fc5 were inserted into the mammalian expression vector pZMP42. Fc5 is an effector minus form of human gamma1 Fc (FIGS. 1A-1B). pZMP42 is a derivative of plasmid pZMP21, made by eliminating the hGH polyadenylation site and SV40 promoter/dhfr gene and adding an HCV IRES/dhfr to the primary transcript, making it tricistronic. pZMP21 is disclosed in US patent application publication No. 2003/0232414 A1 and has been deposited at the American Type Culture Collection, 10801 University Boulevard, Manassas, Va. 20110-2209, designated No.PTA-5266.

The indicated fragment of IL27RA cDNA (nucleotides 23-1558 of SEQ ID NO:2) was isolated using PCR. The upstream primer for PCR (zc53405; SEQ ID NO:11) included, from 5′ to 3′ end, 37 by of flanking sequence from the vector and 21 by corresponding to the amino terminus from the open reading frame of IL27RA. The downstream primer (zc51828; SEQ ID NO:12) consisted of, from 5′ to 3′, 39 by of the bottom strand sequence of Fc5 fusion protein sequence and the last 24 by of the IL27RA extracellular domain sequence, nucleotides 1538 to 1558 of SEQ ID NO:2.

The Fc5 moiety was made with an upstream primer (zc51827; SEQ ID NO:13) including, from 5′ to 3′, 39 by of flanking sequence from the IL27RA extracellular domain sequence and 24 by corresponding to the sequence for the amino terminus of the Fc5 partner. The downstream primer for the Fc5 portion of the fusion protein (zc42508; SEQ ID NO:14) consisted of, from 5′ to 3′, 42 by of the flanking sequence from the vector, pZMP42, and the last 20 by of the Fc5 sequence.

The PCR amplification reaction conditions were 1 cycle, 94° C., 5 minutes; 25 cycles, 94° C., 1 minute, followed by 65° C., 1 minute, followed by 72° C., 1 minute; 1 cycle, 72° C., 5 minutes. Ten μL of each 100-μL PCR reaction mixture was run on a 0.8% low melting temperature agarose gel (SEAPLAQUE GTG) with 1×TBE buffer (0.892M Tris Base, 0.0223M EDTA, 0.890M boric acid) for analysis. The plasmid pZMP42, which had been cut with BglII, was used for homologous recombination with the PCR fragments. The remaining 90 μL of each PCR reaction and 200 ng of cut pZMP42 was precipitated with the addition of 20 μL 3 M Na Acetate and 500 μL of absolute ethanol, rinsed, dried, and resuspended in 10 μL water.

One hundred μL of competent yeast cells (S. cerevisiae) was combined with 10 μL of the DNA mixture from above and transferred to a 0.2-cm electroporation cuvette. The yeast/DNA mixtures were electropulsed at 0.75 kV (5 kV/cm), ∞ ohms, 25 μF. To each cuvette was added 600 μL of 1.2 M sorbitol, and the yeast was plated in two 300-μL aliquots onto two URA-D plates (U.S. Pat. No. 5,736,383) and incubated at 30° C. After about 48 hours, approximately 50 μL packed yeast cells taken from the Ura+ yeast transformants of a single plate was resuspended in 100 μL of lysis buffer (2% TRITON X-100, 1% SDS, 100 mM NaCl, 10 mM Tris, pH 8.0, 1 mM EDTA), 100 μL of resuspension buffer (Buffer P1; QIAGEN Inc., Valencia, Calif.) from a Qiagen miniprep kit (Invitrogen, Carlsbad, Calif.) and 20 U of a B-1,3-glucan laminaripentaohydrolase and b-1,3-glucanase (ZYMOLYASE; Zymo Research, Orange, Calif.). This mixture was incubated for 30 minutes at 37° C., and the remainder of the miniprep protocol (QIAGEN Inc.) was performed. The plasmid DNA was eluted twice in 100 μL water and precipitated with 20 μL 3 M Na Acetate and 500 μL absolute ethanol. The pellet was rinsed once with 70% ethanol, air-dried, and resuspended in 10 μL water for transformation.

Fifty μL electrocompetent E. coli cells (DH10B, Invitrogen, Carlsbad, Calif.) were transformed with 2 μL yeast DNA. The cells were electropulsed at 1.7 kV, 25 μF and 400 ohms. Following electroporation, 1 ml SOC (2% BACTO Tryptone (DIFCO, Detroit, Mich.), 0.5% yeast extract (DIFCO), 10 mM NaCl, 2.5 mM KCl, 10 mM MgCl₂, 10 mM MgSO₄, 20 mM glucose) was plated in 250, 100 and 10 μl aliquots on three LB AMP plates (LB broth (Lennox), 1.8% BACTO Agar (Difco), 100 mg/L Ampicillin).

Individual clones harboring the correct expression construct for IL27RA-Fc5 were identified by restriction digest to verify the presence of the insert and to confirm that the various DNA sequences had been joined correctly to one another. The inserts of positive clones were subjected to sequence analysis. Larger scale plasmid DNA was isolated using a commercially available kit (QIAGEN Maxi kit; QIAGEN Inc., Valencia, Calif.) according to the manufacturer's instructions. DNA and amino acid sequence for IL-27RA-Fc5 are shown in SEQ ID NOS:15 and 16.

Three sets of 200 μg of the IL27RA-Fc5 constructs were separately digested with 200 units of PvuI at 37° C. for three hours, precipitated with ethanol, and centrifuged in a 1.5-mL microfuge tube. The supernatant was decanted off the pellet, and the pellet was washed with 300 μL of 70% ethanol and allowed to incubate for 5 minutes at room temperature. The tube was spun in a microfuge for 10 minutes at 14,000 RPM, and the supernatant was decanted off the pellet. The pellet was then resuspended in 750 μL of CHO cell tissue culture medium in a sterile environment, allowed to incubate at 60° C. for 30 minutes, then allowed to cool to room temperature. Approximately 5×10⁶ CHO cells were pelleted in each of three tubes and resuspended using the DNA-medium solution. The DNA/cell mixtures were placed in a 0.4-cm gap cuvette and electroporated at 950 μF, high capacitance, 300 V. The contents of the cuvettes were then removed, pooled, and diluted to 25 mL with CHO cell tissue culture medium and placed in a 125-mL shake flask. The flask was placed in an incubator on a shaker at 37° C., 6% CO₂ with shaking at 120 RPM.

The CHO cells were subjected to nutrient selection followed by step amplification to 200 nM methotrexate (MTX), and then to 1 μM MTX. Tagged protein expression was confirmed by Western blot, and the CHO cell pool was scaled-up for harvests for protein purification.

To purify the IL27RA-Fc5 fusion protein, 10 L of conditioned media were harvested, sterile filtered using 0.2 μm filters, and adjusted to pH 7.2. The protein was purified from the filtered media using a combination of affinity chromatography on protein A and size-exclusion chromatography. A 117-ml (50 mm×60 mm) protein A column (POROS A50 Applied Biosciences, Foster City, Calif.) was pre-eluted with 3 column volumes (CV) of 25 mM sodium citrate-sodium phosphate, 250 mM ammonium sulfate pH 3 buffer and equilibrated with 20 CV PBS. Direct loading to the column at 31 cm/hr overnight at 4° C. captured the IL27RA-Fc5 protein in the conditioned media. After loading was complete, the column was washed with 10 CV of equilibration buffer. The column was then washed with 10 CV of 25 mM sodium citrate-sodium phosphate, 250 mM ammonium sulfate pH 7.2 buffer, then the bound protein was eluted at 92 cm/hr with a 20 CV gradient from pH 7.2 to pH 3 formed using the citrate-phosphate-ammonium sulfate buffers. Fractions of 10 ml each were collected into tubes containing 500 μl of 2.0 M Tris, pH 8.0 in order to neutralize the eluted proteins. The fractions were pooled based on A₂₈₀ and non-reducing SDS-PAGE.

The IL27RA-Fc5-containing pool was concentrated to 10 ml by ultrafiltration using centrifugal membrane filters (AMICON Ultra-15 30K NWML centrifugal devices; Millipore Corporation, Billerica, Mass.) and injected onto a 318-ml (26 mm×600 mm) size-exclusion chromatography column (SUPERDEX 200 GE Healthcare, Piscataway, N.J.) pre-equilibrated in 35 mM sodium phosphate, 120 mM NaCl pH 7.3 at 28 cm/hr. The fractions containing purified IL27RA-Fc5 were pooled based on A₂₈₀ and SDS PAGE, filtered through a 0.2-μm filter, and frozen as aliquots at ±80° C. The concentration of the final purified protein was determined by colorimetric assay (BCA assay; Pierce, Rockford, Ill.). The overall process recovery was approximately 80%.

Recombinant IL27RA-Fc5 was analyzed by SDS-PAGE (4-12% BisTris, Invitrogen, Carlsbad, Calif.) with 0.1% Coomassie 8250 staining for protein and immunoblotting with Anti-IgG-HRP. The purified protein was electrophoresed and transferred to nitrocellulose (0.2 μm; Invitrogen, Carlsbad, Calif.) at ambient temperature at 600 mA for 45 minutes in a buffer containing 25 mM Tris base, 200 mM glycine, and 20% methanol. The filters were then blocked with 10% non-fat dry milk in 50 mM Tris, 150 mM NaCl, 5 mM EDTA, 0.05% Igepal (TBS) for 15 minutes at room temperature. The nitrocellulose was quickly rinsed, and the IgG-HRP antibody (1:10,000) was added. The blots were incubated overnight at 4° C., with gentle shaking Following the incubation, the blots were washed three times for 10 minutes each in TBS, and then quickly rinsed in H₂O. The blots were developed using commercially available chemiluminescent substrate reagents (LUMILIGHT; Roche), and the signal was captured using commercially available software (Lumi-Imager's Lumi Analyst 3.0; Boehringer Mannheim GmbH, Germany). The purified IL27RA-Fc5 appeared as a band at about 200 kDA on both the non-reducing Coomassie-stained gel and on the immunoblot, suggesting a glycosylated dimeric form as expected. Size-exclusion chromatography/multi-angle light scattering (SEC MALS) confirmed a mass consistent with a dimer containing additional mass contribution from carbohydrate at approximately 27% by weight, for a total mass of 212 kD (+/−5%). The IL27RA-Fc5 polypeptide had the correct NH₂ terminus and the correct amino acid composition.

### Example 3

Whole mouse spleens were harvested from C57 B1/6 mice and washed two times with 1×PBS before being plated out at 2×10⁵ cells/well in assay media (RPMI 1640 plus 10% fetal bovine serum) in 96-well, round-bottom tissue culture plates. Total human PBMC (peripheral blood mononuclear cells) were thawed from a frozen vial collected from a leukapherisis donation and washed two times with 1×PBS before being plated out at 10⁶ cells/well in assay media in 96-well, round-bottom tissue culture plates. A sub-maximal concentration (EC₉₀, effective concentration at 90 percent) of mouse IL-27 and human IL-27 (R&D Systems, Minneapolis, Minn.) were each combined with a dose range of the human IL-27RA and mouse IL-27RA soluble receptors and incubated together at 37° C. for 30 minutes in assay media prior to addition to cells. Following pre-incubation, treatments were added to the plates containing the cells and incubated together at 37° C. for 15 minutes.

Following incubation, cells were washed with ice-cold wash buffer (BIO-PLEX Cell Lysis Kit, BIO-RAD Laboratories, Hercules, Calif.) and put on ice to stop the reaction according to manufacturer's instructions. Cells were then spun down at 2000 rpm at 4° C. for 5 minutes prior to dumping the media. 50 μL/well lysis buffer was added to each well; lysates were pipetted up and down five times while on ice, then agitated on a microplate platform shaker for 20 minutes at 300 rpm and 4° C. Plates were centrifuged at 4500 rpm at 4° C. for 20 minutes. Supernatants were collected and transferred to a new microtiter plate for storage at −20° C.

Capture beads (BIO-PLEX Phospho-Stat3 Assay, BIO-RAD Laboratories) were combined with 50 μL of 1:1 diluted lysates and added to a 96-well filter plate according to manufacture's instructions (BIO-PLEX Phosphoprotein Detection Kit, BIO-RAD Laboratories). The aluminum foil-covered plate was incubated overnight at room temperature with shaking at 300 rpm. The plate was transferred to a microtiter vacuum apparatus and washed three times with wash buffer. After addition of 25 μL/well detection antibody, the foil-covered plate was incubated at room temperature for 30 minutes with shaking at 300 rpm. The plate was filtered and washed three times with wash buffer. Streptavidin-PE (50 μL/well) was added, and the foil-covered plate was incubated at room temperature for 15 minutes with shaking at 300 rpm. The plate was filtered and washed two times with bead resuspension buffer. After the final wash, beads were resuspended in 125 μL/well of bead suspension buffer, shaken for 30 seconds, and read on an array reader (BIO-PLEX, BIO-RAD Laboratories) according to the manufacture's instructions. Data were analyzed using analytical software (BIO-PLEX MANAGER 3.0, BIO-RAD Laboratories). Decreases in the level of the phosphorylated STAT3 transcription factor present in the lysates were indicative of neutralization of the IL-27 receptor-ligand interaction.

For mouse spleens, muIL-27 EC₉₀ concentration was determined to be 0.2 nM and huIL-27 to be 2 nM. For total human PBMCs, both mouse and human IL-27 EC₉₀ concentrations were 2 nM. Run in combination with a dose-response of the muIL-27RA or huIL-27RA soluble receptor, the IC₅₀ (inhibitory concentration at 50%) was determined for each soluble receptor to each ligand on both cell types. Data are shown in Tables 1 and 2.

TABLE 1
Mouse Spleens

| Ligand | Soluble Receptor | IC₅₀ (nM) |
|---|---|---|
| muIL-27 | IL-27RAm(mFc1) | 0.18 |
| muIL-27 | human IL-27RA-Fc5 | 0.14 |
| huIL-27 | IL-27RAm(mFc1) | 9.30 |
| huIL-27 | human IL-27RA-Fc5 | 0.32 |

TABLE 2
Total Human PBMCs

| Ligand | Soluble Receptor | IC₅₀ (nM) |
|---|---|---|
| muIL-27 | IL-27RAm(mFc1) | 4.83 |
| muIL-27 | human IL-27RA-Fc5 | 2.97 |
| huIL-27 | IL-27RAm(mFc1) | 1370 |
| huIL-27 | human IL-27RA-Fc5 | 0.95 |

### Example 4

Kinetic rate and affinity constant values for the mouse (IL27RAm(mFc1), Example 1) and human (IL27RA-Fc5, Example 2) soluble receptors were obtained by surface plasmon resonance (SPR) using an automated instrument (BIACORE 3000; Biacore International AB, Uppsala, Sweden). The mouse soluble receptor was tested against mouse ligand (lot A1418F), and the human soluble receptor was tested against both mouse (A1426F) and human (A1534F) ligands. For determination of the kinetic rate constants for the receptor-ligand interactions, the gp130 molecule was not included as part of the receptor complex. Experimental evidence indicated that gp130 did not play a role in the binding mechanism, but affected only signaling (i.e., subsequent generation of physiological response), hence the measurement of the interaction between IL27RA and IL27 ligand was expected to accurately assess the affinity of simple binding of the ligand to its receptor.

The IL27 ligands used in this study were single-chain molecules comprising EBI3 connected by its C-terminus to the N-terminus of IL-27 p28 via a polypeptide linker. (R&D Systems) Each of the ligands included an amino-terminal peptide tag.

For the mouse IL27RA study, the soluble receptor was captured onto the chip surface by an isotype-specific anti-mouse Fc antibody (obtained from Jackson ImmunoResearch Laboratories, Inc., West Grove, Pa.) covalently immobilized to the chip (BIACORE CM5 chip) using the standard amine coupling protocol specified by the instrument manufacturer. For the human IL27RA studies, the soluble receptor was directly and covalently immobilized to the chip via the amine coupling protocol. In all studies, ligand was injected over the active (receptor-bound) surface at varying concentrations to obtain a series of binding curves.

Experimental conditions were optimized for determination of kinetic rate constant values. The molecular densities of the soluble receptor protein loaded onto the chip surface were targeted to obtain maximum IL27 binding levels (Rₘₐₓ) of ≦20 RU. The analyte (ligand) was injected over the receptor surface at a flow rate of 50 4/minute at a concentration range of approximately 0.05 to 10 nM, allowing for an association phase of 3 minutes and a dissociation phase of 10 minutes. The mouse soluble receptor surface was regenerated with two 30-second injections at 50 μL/minute of glycine, pH 2.0. The human soluble receptor surface was similarly regenerated with a single 30-second injection.

All data were assessed using software provided with the instrument (BIACORE Evaluation software v. 3.2). The binding curves were globally fitted to a 1:1 binding model corrected for mass transport limitation resulting from the fast on-rate values (kₐ) obtained. Statistical analysis of the fits of the experimental binding curves versus theoretical curves gave standard error values for kₐ and k_{d} of less than 2%, and chi² values of less than 2% of Rₘₐₓ for all interactions tested, providing reasonable confidence in the kinetic rate constant values obtained.

The kinetic rate and affinity constants obtained for mouse soluble receptor binding with mouse ligand were kₐ=1.0×10⁷ (M⁻¹s⁻¹), k_{d}=1.2×10⁻³ (s⁻¹) and K_{d}=1.2×10⁻¹⁰M (K_{d}=k_{d}/kₐ). The kinetic rate and affinity constants obtained for human soluble receptor binding with human ligand were kₐ=1.0×10⁷ (M⁻¹s⁻¹), k_{d}=1.9×10⁻³ (s⁻¹) and K_{d}=1.9×10⁻¹⁰ M. The kinetic rate and affinity constants obtained for human soluble receptor binding with mouse ligand were kₐ=8.1×10⁶ (M⁻¹s⁻¹), k_{d}=1.8×10⁻³ (s⁻¹) and K_{d}=2.2×10⁻¹⁰ M.

### Example 5

Studies were performed to evaluate the pharmacokinetics of the mouse (IL-27RAm(mFc1)) and human (IL-27RA-Fc5) soluble receptors in female C57B1/6 mice. Mice were randomly assigned to treatment groups as shown in Table 3.

TABLE 3

| | Route of | Dose | Sample Time Points |
|---|---|---|---|
| Treatment | Admin. | (μg) | (hours postdose) |
| IL- | IV | 100 | 0.25, 1, 3, 6, 24, 48, & 120 |
| 27RAm(mFc1) | IP | 100 | |
| | SC | 100 | |
| IL-27RA-Fc5 | IV | 100 | 0.25, 0.5, 1, 3, 6, 24, 48, & 120 |
| | IP | 100 | |
| | SC | 100 | |

Whole blood was collected at the time points listed in Table 3. Serum was generated from each sample and analyzed by a qualified enzyme-linked immunosorbant assay (ELISA). The resulting mean serum concentration versus time profiles were then subjected to noncompartmental pharmacokinetic analyses. The following pharmacokinetic parameters were calculated: C₀ and Cₘₐₓ (extrapolated concentration at time zero and maximum serum concentration, respectively), Tₘₐₓ (time to achieve maximum concentration), t_{1/2 λz} (terminal half-life), AUC₀₋ₜ (area under the concentration versus time curve from time zero to the last measurable time point), AUC_{INF} (area under the concentration versus time curve extrapolated to infinity), C1 or C1/F (clearance or clearance divided by bioavailable fraction, respectively), V_{SS} or V_{Z}/F (steady state volume of distribution or volume of distribution divided by the bioavailable fraction, respectively), and F (bioavailable fraction). Results are summarized in Table 4.

TABLE 4

| Treatment | Parameter | Units | IV | IP | SC |
|---|---|---|---|---|---|
| IL-27RAm | Cₒ; Cₘₐₓ | μg/mL | 36.2 | 13.2 | 7.95 |
| (mFc1) | Tₘₐₓ | h | — | 1 | 3 |
| | AUC₀₋₂₄ | h * μg/mL | 157 | 98.2 | 56.5 |
| | AUC_{INF} | h * μg/mL | 162 | 102 | NE |
| | t_{1/2 λz} | h | 5.25 | 5.17 | NE |
| | Vₛₛ; V_{z}/F | mL | 3.05 | 7.33 | NE |
| | Cl; Cl/F | mL/h | 0.618 | 0.983 | NE |
| | F | — | — | 0.625 | 0.360 |
| IL-27RA- | Cₒ; Cₘₐₓ | μg/mL | 122 | 35.2 | 21.0 |
| Fc5 | Tₘₐₓ | h | — | 3 | 6 |
| | AUC₀₋₁₂₀ | h * μg/mL | 1400 | 1310 | 1130 |
| | AUC_{INF} | h * μg/mL | 1510 | 1380 | 1210 |
| | t_{1/2 λz} | h | 34.9 | 28.3 | 29.3 |
| | Vₛₛ; V_{z}/F | mL | 2.54 | 2.96 | 3.50 |
| | Cl; Cl/F | mL/h | 0.0664 | 0.0725 | 0.0828 |
| | F | — | — | 0.914 | 0.801 |

NE, not estimable due to an insufficient characterization of the terminal portion of the concentration versus time curve;
—, not applicable.

In summary, the human Fc5 fusion protein was found to have a much longer terminal half-life (t_{1/2 λz} than the mouse Fc1 fusion protein. This difference in t_{1/2 λz} between the two proteins is due to a more rapid clearance of IL-27RAm(mFc1) compared to IL-27RA-Fc5.

### Example 6

IL27-transgenic mice were produced by inter-crossing IL-27 p28 single-transgenic mice with EBI3 single-transgenic mice. The cDNAs for mouse EBI3 and IL-27 p28 were cloned into a vector under the control of the mouse LCK proximal promoter with the mouse E.mu. heavy-chain enhancer (Iritani et al., EMBO 16: 7019-7031, 1997). In transgenic mice, expression of this promoter/enhancer is primarily in B and T cells starting at approximately day 13 of embryonic development. The constructs were injected into B6C3F1 fertilized embryos, and three lines were established for each construct from identified founders (generation N0) by breeding single-transgenics with C57BL/6N mice. Second generation (N2) EBI3 and IL-27 p28 single-transgenic mice were cross-bred to produce double-transgenic offspring (hereinafter referred to as “IL27 transgenics”). Cross-breeding produced litters with a Mendelian distribution of double-transgenic, single-transgenic, and wild-type pups. Transgene expression was monitored by PCR of the hGH poly-A region present in both constructs.

Levels of cytokines in serum from IL27-transgenic and wild-type littermate mice, all 3-6 weeks of age, were measured using a commercially available kit (Luminex Corporation, Austin, Tex.). The transgenic mice had significantly higher levels of inflammatory cytokines in their serum than did their wild-type littermates.

Immune cells in spleen, thymus and bone-marrow from 3 week-old IL27-transgenic mice and their littermates were analyzed by 8-color flow cytometry. Spleen and thymus cells were stained for CD44, CD62L, CD69, CD3, CD8, CD49, CD25, and CD4 to identify T cell subpopulations, NKT cells, and NK cells. Thymus and spleen cells from 3 week-old IL27-transgenic mice and their littermates were also stained with antibodies against cell-surface CD4, CD8, CD25, and intracellular FoxP3 to identify regulatory T cells (T_{reg}). Spleen cells were stained for CD23, CD21, CD11b, IgM, IgD, CD11c, Gr-1, and B220 to identify B cell subpopulations, granulocytes, macrophages, and dendritic cells. Bone marrow cells were stained for IgD, CD43, CD11b, IgM, B220, CD11c, and Gr-1 to identify B cell subpopulations, macrophages, dendritic cells, and granulocytes. IL27-transgenic mice had significantly fewer B cells, NK cells, and naïve T cells; increased numbers of macrophages, granulocytes, and activated/memory T cells; and lacked T_{reg} cells in lymphoid tissues. T_{reg} were defined as being CD4⁺CD8⁻ and FoxP3⁺.

CD4⁺ and CD8⁺ T cells were purified from spleens of 5 week-old IL27-transgenic and wild-type mice using superparamagnetic particles coupled to monoclonal antibodies (MACS beads; Miltenyi Biotec Inc., Auburn, Calif.). The T cells (5×10⁵/well) were stimulated in flat-bottom 96-well plates with plate-bound anti-CD3 mAb (5.micro.g/ml), irradiated BALB/c spleen cells (5×10⁶/well) or medium alone. Cell supernatants were collected at 48 hours for determination of cytokine levels using a commercially available kit (Luminex Corporation, Austin, Tex.). Proliferation ([³H]-thymidine incorporation) of triplicate cultures was quantitated at 72 hours using a beta counter. CD4⁺ T cells from the IL27 transgenics displayed decreased effector-function compared to wild-type cells upon in vitro stimulation, whereas CD8′ T cells displayed increased effector-function.

Immunohistochemistry (IHC) analysis of tissue sections of 4-6 week old mice showed that the IL27-transgenics had multi-organ inflammation (affecting liver, lung, pancreas, GI mucosa, and kidney), with mild-moderate lymphocytic infiltrates in multiple tissues. Infiltrates were perivascular, peribronchial, or interstitial and were comprised mostly of F4/80′ macrophages plus some T cells. Lymphoid depletion was observed in tissue sections of spleen, lymph nodes and intestine (Peyer's patches).

IL27-transgenic mice became cachectic and moribund between 5-10 weeks of age.

The observed phenotype of the IL27-transgenic mice was similar to the systemic inflammatory response observed in acute GVHD. In particular, the mice exhibited (1) multi-organ inflammatory infiltrate comprised mostly of macrophages/dendritic cells and T cells; (2) tissue damage, particularly to liver, gut and skin; (3) elevated levels of inflammatory cytokines (TNF-alpha, IL-6, IL-1, IL-10, IFN-gamma) in the serum; (4) increased numbers of activated CD8 T cells that produce significant amounts of IFN-□, TNF.alpha. and IL-10 and have cytotoxic activity against host cells; and (5) defects in hematapoesis.

### Example 7

An experiment was conducted to determine whether hydrodyamic delivery of IL-27 into normal adult mice caused the same immune phenotype as was observed in IL-27 transgenic mice. A single-chain mouse IL-27 sequence (encoding EBI3 and p28 joined by a short linker) was inserted into the polylinker of the vector PLIVE (Minis Bio Corporation, Madison, Wis.), which is a liver-directed expression vector comprising a mouse minimal albumin promoter and mouse alpha fetal protein enhancer. C57BL/6 female mice (age approximately 8 weeks) were divided into four groups, which received either nothing (untreated; n=5), PBS (n=8), 20.micro.g empty-vector pLIVE DNA (n=8), or 20.micro.g pLIVE-IL27 DNA (n=8) via tail vein injection. Plasmid DNAs were diluted to 10.micro.g/ml in sterile saline and delivered i.v. in 2 ml volume/mouse. On days 2 and 7 after injection, serum was collected from half of the mice by eye-bleed. Mice bled on day 2 were sacrificed on day 16, and mice bled on day 7 were sacrificed on day 21. Splenectomy was performed, then serum was collected by cardiac puncture. Spleen, thymus, and bone marrow were collected for FACS analysis. All tissues were placed in individual tubes containing sterile PBS, on ice. Serum was analyzed for levels of IL-27 by ELISA, and inflammatory cytokines were analyzed by bead array using commercially available reagents (UpState Cell Signaling Solutions, Temecula, Calif.). Serum samples from each time-point were also pooled by group and sent for analysis by Rules-Based Medicine (Austin, Tex.).

In summary, hydrodynamic delivery of IL-27 into normal adult mice had the same immune effects as were seen in IL27-transgenic mice and mice infected with IL27-Adenovirus. These effects included downregulation of CD25 and FoxP3 expression by regulatory T cells (CD4+FoxP3+). At days 16 and 21, spleens of mice that received IL-27 exhibited an increased (2×) number of total splenocytes and CD4+ cells; a decreased (3×) ratio of CD25+ to CD25-cells among Treg (CD4+FoxP3+ cells); and decreased (2×) levels of FoxP3 and CD25, but not GITR (glucocorticoid-induced tumor necrosis factor receptor family-related gene), expressed by Treg. Additional findings, and a comparison with IL27 transgenic and IL27-adenovirus mice, are shown in Table 5.

TABLE 5

| | | | Transgenic |
|---|---|---|---|
| | pLIVE mIL-27 HDD | Adenovirus mIL-27 | zalpha51/EBI3 |
| Body Weight | No significant changes | No significant | Transgenics have rapid |
| | | changes | decline in body weight |
| Spleen Weight | Spleen larger | Spleen larger | Spleen small |
| Thymus Weight | No Change | Thymus smaller | Thymus small |
| Serum | Possible decrease in IL-2 | Possible increases in | Increased IL-5, IL-6, |
| Cytokines | | IL-6 | TNF\acute{α}, IFNγ, and IL-10 |
| | | TNF\acute{α} & IFNγ | |
| | | similar to PBS treated | |
| | | mice. | |
| Serum Ig | No data | Adeno-specific | Normal levels of |
| Isotypes | | antibody is | IgG2a; All others |
| | | predominantly IgG2a | significantly decreased |
| Cell | Not tested | Lower CD4+ T cell | Lower T cell |
| Proliferation | | proliferation | proliferation |
| | | B cell proliferation | Lower B cell |
| | | not tested | proliferation |
| B cells | BM lacks mature & | BM lacks mature & | BM lacks mature & |
| | new B cells. | new B cells. | new B cells |
| | Decreased B cells in | Decreased B cells in | Spleen has no mature |
| | spleen by day 21 | spleen by day 21 | B cells |
| | No data for PEC | No data for PEC | PEC has no B-1 B cells |
| T Cells | Thymus involuted | Thymus involuted | Thymus involuted |
| | Increase in activated | Increase in activated | Increase in activated |
| | cells; Decrease in naïve | cells; Decrease in | cells; Decrease in |
| | cells. | naïve cells. | naïve cells. |
| Treg | Decrease in CD25 MFI | Decrease in CD25 | No CD25 MFI |
| | | MFI | |
| | Decrease in | Decrease in | Absence of |
| | CD4+FoxP3+ T cells | CD4+FoxP3+ T cells | CD4+FoxP3+T cells |
| NK Cells | No changes | Decreased early on | Absent |
| Macs/Grans/DC | Increased spleen, BM | Increased spleen & | Increased spleen & |
| | and LN Macs, DCs, | BM monocytes, DCs, | BM monocytes, DCs, |
| | and granulocytes | and granulocytes | and granulocytes |
| Viability | All mice survived | All mice survived | Low expressing line |
| | experiments | experiments | viable, High |
| | | | expressing line dies |
| | | | ~5-10 weeks |

Abbreviations:
BM, bone marrow;
PEC, peritoneal exudate cells, collected by rinsing the peritoneal cavity with PBS;
MFI, mean flourescence intensity of population as measured by flow cytometry;
Macs, macrophages;
Grans, granulocytes;
DC, dendritic cells;
LN, lymph node.

### Example 8

A second hydrodynamic delivery experiment was conducted to determine whether IL-2 treatment of mice overexpressing IL-27 could increase expression of CD25 and FoxP3 on regulatory T cells (Treg). Treatment of mice with recombinant IL-2 has been shown to upregulate CD25 and FoxP3 expression by Treg in vivo (Fontenot et al., Nature Immunology 6(11):1142-1151, 2005).

C57BL/6 female mice (age approximately 8 weeks) were divided into four groups of four mice each. Mice in groups 1 and 2 received empty-vector pLIVE DNA (20.micro.g in 2 ml sterile saline), and mice in groups 3 and 4 received pLIVE-IL27 DNA (20.micro.g in 2 ml sterile saline) via tail vein injection. Mice were treated by intraperitoneal injection with 100.micro.1/mouse of either diluent (groups 1 and 3) or recombinant human IL-2 (R&D Systems, Inc.; Minneapolis, Minn.; diluted to 10.micro.g/ml in PBS) (groups 2 and 4) on days 13-14 (total of four injections at 8-hour intervals). One hour after the last injection, the mice were sacrificed. Splenectomy was performed, then serum was collected by cardiac puncture. Spleen and thymus were collected for FACS analysis. Tissues were placed in individual tubes containing sterile PBS, on ice. Serum was analyzed for levels of IL-27 by ELISA.

Mice overexpressing IL-27 did not increase spleen cell numbers in response to IL-2. Administration of IL-2 did not result in significant differences in thymic T-cells or in splenic NKT cells, NK cells, B cells, macrophages, granulocytes, or dendritic cells. Splenic Treg exposed to IL-27 did not increase FoxP3 or CD25 expression in response to IL-2. In summary, IL-2 was not able to reverse the effects of IL-27 overexpression on Treg. The data suggest that IL-27 renders Treg unresponsive to IL-2.

### Example 9

Efficacy of IL-27 antagonists was assayed in a mouse model of acute graft-vs-host disease (Durie et al., J. Clin. Invest. 94:1333-1338, 1994). Parental mice (C57BL/6; n=12) were euthanized, and their spleens were collected. The pooled spleens were smashed using two glass slides to dissociate splenic cells. Lysis buffer was added to the splenocyte suspension to remove red blood cells. The cells were washed in RPMI 1640 (10% FBS) medium and resuspended in an appropriate amount of PBS to make a cell concentration of 300 million cells/ml. Recipient mice (C57BL/6×DBA/2 F1) were divided into treatment groups as shown in Table 6. Protein treatments were administered by intraperitoneal injection every other day beginning on day-1 and continuing until day 15. Dexamethasone (DEX) was administered by injection daily on days 0 through 6. On day 0, 75 million donor splenic lymphocytes from B6 mice (250.micro.1 per injection) were injected intravenously into recipient mice (C57BL/6×DBA/2 F1 (BDF1); n=10 per group) mice. Mice were monitored 3 times a week for changes in body weight and any signs of moribundity. Mice that lost >20% of their initial body weight were euthanized. Otherwise, mice were sacrificed 18 days after the cell transfer. Spleens were collected, and a CTL-specific lysis assay using P815 cells was performed as a quantitative measurement of acute GVHD. Furthermore, spleens were stained for T- and B-cell markers, including MHC class I markers (H2^{b} and H2^{d}) to look at donor/recipient cell ratio (acute GVHD spleen cells are mostly donor cells). Sera were collected to measure serum level of IgG1, IgG2a, and IgE by ELISA, and cytokine and chemokine levels using a commercially available kit (Luminex Corporation, Austin, Tex.).

TABLE 6

| Group | n | Treatment |
|---|---|---|
| PBS | 10 | PBS, 100 .micro.l/dose |
| IL27RA Fc5 | 10 | Human IL-27RA-Fc5, 1 mg/ml, 100 .micro.l/dose |
| Anti-IL27RA | 10 | Anti-mouse IL-27RA monoclonal antibody, |
| mAb | | 1 mg/ml, 100 .micro.l/dose |
| DEX (+control) | 8 | Dexamethasone, 400 .micro.g/ml, 100 .micro.l/dose |

For CTL assay, P815 cells (a tumor cell line from mice with the same MHC class as DBA2) were labeled with calcein, then splenocytes from each experimental animal were added to the calcein-labeled P815 cells at effector (splenocytes):target (P815) ratios of 100:1, 33:1, and 10:1. Four hours after incubation at 37° C., supernatants were collected and fluorescence was measured (485 nm/535 nm).

Results of the study showed a correlation of the animal model with development of acute GVHD. There was a loss of host (BDF1) spleen cells and decreased numbers of donor (C57B1/6) Treg cells in PBS controls. In the treated animals, both the IL27RA-Fc5 polypeptide and an anti-IL27RA mAb maintained host spleen cells, CD4+T cells, and Treg cells. In contrast, dexamethasone treatment did not maintain host spleen cells, CD4+T cells, or Treg cells. No treatment prevented the activation or expansion of donor (C57B1/6) conventional CD4+T cells. All groups had similar numbers of donor conventional CD4+T cells, and GITR (glucocorticoid-induced tumor necrosis factor receptor family-related gene) was upregulated by donor conventional CD4+T cells. Body weight loss in both of the IL-27RA-Fc and the anti-IL-27RA mAb treatment groups was not severe and was significantly less than in the PBS control group. IL-27RA-Fc polypeptide and anti-IL-27RA mAb did not prevent splenomegaly, but did prevent colon length shortening. IL-27RA-Fc polypeptide and anti-IL-27RA mAb treated animals show reduced CTL activity compared to the PBS group. The effects of the IL-27RA-Fc polypeptide and the anti-IL-27RA mAb treatments on immunoglobulin and cytokine levels are shown in Table 7.

TABLE 7

| | | | IL-27RA |
|---|---|---|---|
| | Control | IL-27RA-Fc | mAb |
| IgG1 | — | ↑↑ | ↑↑↑ |
| IgG2A | ↑ | ↑↑ | ↑↑ |
| IgE | ↓ | ↑ | ↑↑↑ |
| IL-2 | ↑ | ↑↑↑ | ↑↑↑ |
| IL-5 | ↑ | ↑↑ | ↑↑ |
| IL-6 | ↑↑↑ | ↓↓↓ | ↓↓↓ |
| IL-10 | ↑↑ | — | ↓↓ |

### Example 10

Naïve T-cells were isolated from the spleens of 6 week-old female BALB/c mice (n=5) using a commercially available kit (CD4⁺ CD62L⁺ T Cell Isolation Kit, mouse; Miltenyi Biotec, Auburn, Calif.). Tissue culture plates were coated with anti-CD3 monoclonal antibody (mAb) (2.0.micro.g/ml in PBS) for 2-4 hours The plates are then washed with PBS to remove unbound anti-CD3. Naïve T cells (4×10⁵/well) were then added to the plates along with anti-CD28 mAb (0.5.micro.g/ml) and single-chain mouse IL-27 (0, 1.1, 3.3, 10, and 30 ng/ml). The cells were then inclubated at 37° C. Cell supernatants were collected from one set of plates at 48 hours and from a duplicate set of plates at 72 hours. The supernatants were stored frozen at −80° C. The IL-2 concentration in each supernatant was measured using a bead-based ELISA assay (LUMINEX; Upstate, Charlottesville, Va.) following the manufacturer's instructions. The data (Table 8) showed that IL-27 inhibited IL-2 production by naive CD4 T cells.

TABLE 8
IL-2 Conc. (pg/ml)

| | | | | antiCD3 + | antiCD3 + | antiCD3 + | | | |
|---|---|---|---|---|---|---|---|---|---|
| Conc. | Null | Null | Null | antiCD28 | antiCD28 | antiCD28 | antiCD3 | antiCD3 | antiCD3 |
| mIL-27 | 24 hr | 48 hr | 72 hr | 24 hr | 48 hr | 72 hr | 24 hr | 48 hr | 72 hr |
| 30 ng/ml | 0 | 0 | 0 | 78.25 | 365.05 | 473.38 | 59.02 | 345.33 | 230.42 |
| 10 ng/ml | 0 | 0 | 0 | 85.25 | 414.91 | 799.85 | | | |
| 3.3 ng/ml | 0 | 0 | 0 | 77.14 | 230.19 | 722.63 | | | |
| 1.1 ng/ml | 0 | 0 | 9.69 | 72.98 | 744.27 | 2785.56 | | | |
| 0 ng/ml | 3.64 | 9.69 | 0 | 45.29 | 574.39 | 3266.41 | 30.64 | 2209.38 | 311.36 |

In a second experiment, naïve CD4 T-cells were isolated as described above. These T cells were then incubated in culture medium with either a neutralizing rat anti-mouse IL-27RA mAb (clone 290.118.6; 100, 30, 10, 3, 1, or 0.micro.g/ml), a rat IgG2a isotype control mAb (100, 30, 10, 3, 1, or 0.micro.g/ml) that does not recognize any mouse protein (obtained from eBioscience, San Diego, Calif.) or no antibody for 30 minutes at 37 degrees C. Tissue culture plates were coated with anti-CD3 mAb as described above. The cells+mAb were then transferred to the anti-CD3 coated assay plates. IL-27 (10 ng/ml) and anti-CD28 (0.5.micro.g/ml) were then added to the cells in the assay plates. The assay plates were inclubated at 37° C. for 48 hours and 72 hours (two sets of plates were prepared one set for each time-point). The supernatants were stored frozen at −80° C. The IL-2 concentration in each supernatant was measured using a bead-based ELISA assay (LUMINEX; Upstate, Charlottesville, Va.) following the manufacturer's instructions. The data (Table 9) confirmed that IL-27 inhibited IL-2 production by naïve CD4 T cells and also showed that this activity of IL-27 could be blocked by a neutralizing rat anti-mouse IL-27RA monoclonal antibody.

TABLE 9
IL-27 effects on CD28 induced IL-2 with IL-27RA mAb
IL-2 concentration, pg/ml

| | | | | |
|---|---|---|---|---|
| | No IL-27 + | | | |
| mAb | IL- | | IL-27 + | |
| conc. | 27RA | No IL-27 + | IL-27RA | IL-27 + Iso |
| (ug/ml) | mAb | Iso cntr | mAb | cntr |

48 hr

| | | | | |
|---|---|---|---|---|
| 0 | 322.48 | 177.76 | 139.94 | 150.29 |
| 1 | 445.08 | 290.95 | 249.96 | 149.1 |
| 3 | 459.38 | 265.83 | 395.09 | 126.95 |
| 10 | 451.43 | 252.15 | 537.14 | 133.62 |
| 30 | 430.73 | 388.26 | 543.84 | 155.42 |
| 100 | 154.69 | 84.49 | 126.9 | 104.54 |

72 hr

| | | | | |
|---|---|---|---|---|
| 0 | 3512.56 | 3035.2 | 179.97 | 217.81 |
| 1 | 2977.34 | 2632.74 | 389.24 | 210 |
| 3 | 2756.21 | 2352.77 | 656.86 | 323.98 |
| 10 | 4417.19 | 3055.18 | 913.41 | 173.61 |
| 30 | 2984.47 | 2781.83 | 1575.23 | 236.31 |
| 100 | 1260.13 | 1438.2 | 1129.74 | 361.08 |

The ability of neutralizing rat-anti-mouse IL-27RA monoclonal antibodies (clones 290.118.6, 290.267.1, 295.6.4, 295.13.4, 295.16.2 and 295.20.4), a mouse soluble receptor (IL27RAm(mFc1)) and a human soluble receptor (IL27RA-Fc5) to block the ability of mouse single-chain IL-27 to inhibit IL-2 production by naïve CD4 T cells was tested in a third set of experiments. Naïve mouse CD4 T-cells were isolated as described above. For testing of the neutralizing mAbs, the naïve CD4 T cells were preincubated for 30 minutes at 37 degrees C. with graded concentrations (60, 30, 15, 7.5, 3.75, 1.875.micro.g/ml) of either neutralizing rat anti-mouse IL27RA mAb (each mAb tested separately), rat IgG1 isotype control mAb, rat IgG2a isotype control mAb, or no mAb. The isotype control mAbs (purchased from eBioscience) do not recognize any mouse protein. The CD4 T cells (4×10⁵/well) were then transferred to anti-CD3 coated tissue culture plates. Single-chain mouse IL-27 (10 ng/ml) was then added to the plates. Duplicate plates were set up for all experimental conditions. The plates were then cultured at 37 degrees for up to 72 hours.

For testing of the soluble receptor, single-chain mouse IL-27 (10 ng/ml) was preincubated for 30 minutes at 37 degrees C. with either IL27RAm(mFc1) (10.0 and 5.0.micro.g/ml), IL27RA-Fc5, mouse Fc1 protein (10.0 and 5.0.micro.g/ml), human Fc5 protein (10.0 and 5.0.micro.g/ml), or no recombinant protein in wells of the anti-CD3 coated plates. 4×10⁵ CD4⁺ CD62L high cells were added to the wells. Duplicate plates were set up for all experimental conditions. The plates were then cultured at 37 degrees for up to 72 hours. Cell supernatants were collected from one set of plates at 48 hours and from the duplicate set of plates at 72 hours. The supernatants were stored frozen at −80° C. The IL-2 concentration in each supernatant was measured using a bead-based ELISA assay (LUMINEX; Upstate, Charlottesville, Va.) following the manufacturer's instructions. The data (Tables 10 and 11) showed that both the mouse and human soluble receptors could partially (−50% inhibition) block the ability of mouse IL-27 to inhibit IL-2 production by naïve CD4 T cells. The soluble receptors were more effective at blocking the activity of IL-27 than were the neutralizing IL-27RA-specific mAbs.

TABLE 10
IL-27 Inhibition of IL-2 production neutralized by IL-27RA Fc
IL-2 concentration (pg/ml)

| Protein | | | | | | |
|---|---|---|---|---|---|---|
| conc. | | 48 hr | | | | |
| (Fc | | Stim + | | | | |
| controls | | mouse | | 48 hr | | 48 hr |
| in | 48 hr | IL-27 | 48 hr | hIL- | 48 hr | mIL- |
| molar | No Add | Only | Fc5 + | 27RA + | Fc1 + | 27RA + |
| equiv.) | (Avg.) | (Avg.) | IL-27 | IL-27 | IL-27 | IL-27 |
| 0 | 10448.28 | 1962.91 | 1777.78 | 1777.19 | 1945.77 | 1256.05 |
| 5 | | | 1489.62 | 5784.11 | 1193.01 | 4021.15 |
| 10 | | | 1715.92 | 5774.94 | 1266.5 | 5215.59 |
| 10 | | | | 5333.6 | | 4922.26 |
| Protein | | | | | | |
| conc. | | 72 hr | | | | |
| (Fc | | Stim + | | | | |
| controls | | mouse | | 72 hr | | 72 hr |
| in | 72 hr | IL-27 | 72 hr | hIL- | 72 hr | mIL- |
| molar | No Add | Only | Fc5 + | 27RA + | Fc1 + | 27RA + |
| equiv.) | (Avg.) | (Avg.) | IL-27 | IL-27 | IL-27 | IL-27 |
| 0 | 717438.2 | 797.9933 | 1142.27 | 1261.82 | 257.52 | 233.69 |
| 5 | | | 1037.11 | 1869.62 | 333.99 | 5744.95 |
| 10 | | | 1001.04 | 28607.63 | 284.59 | 6704.62 |
| 10 | | | | 17481.17 | | 12186.7 |

TABLE 11
IL-27 Inhibition of IL-2 production neutralized by IL-27RA mAbs
IL-2 concentration (pg/ml)

| | | 48 hr | | | | | | | | |
|---|---|---|---|---|---|---|---|---|---|---|
| | | Stim + | | | | | | | | |
| | | mouse | | | | | | | | |
| | 48 hr | IL-27 | 48 hr | 48 hr | 48 hr | 48 hr | 48 hr | 48 hr | 48 hr | 48 hr |
| mAb | No Add | Only | IgG1 + | IgG2a + | E9633 + | E9630 + | E9631 + | E9629 + | E9632 + | E9518 + |
| conc. | (Avg.) | (Avg.) | IL-27 | IL-27 | IL-27 | IL-27 | IL-27 | IL-27 | IL-27 | IL-27 |
| 0 | 10448.28 | 1962.91 | 996.03 | 731.43 | 1074.71 | 513.88 | 843.48 | 922.8 | 897.81 | 802.26 |
| 1.875 | | | 984.32 | 1080.75 | 1570.85 | 1036.13 | 1098.78 | 1343.28 | 1408.87 | 1045 |
| 3.75 | | | 1042.24 | 885.98 | 2060.28 | 1180.62 | 1227.42 | 1308.03 | 1603.96 | 1208.95 |
| 7.5 | | | 1094.51 | 1040.85 | 2350.82 | 981.07 | 1416.35 | 1268.96 | 1489.12 | 1198.17 |
| 15 | | | 993.14 | 913.2 | 2957.9 | 726.08 | 2158.48 | 1343.75 | 1707.16 | 1575.58 |
| 30 | | | 992.95 | 1099.19 | 3343.45 | 944.2 | 2215.71 | 1475.59 | 2383.51 | 1219.36 |
| 60 | | | 878.56 | 991.61 | 2931.81 | 1173.82 | 2373.34 | 1812.58 | 2699.16 | 1140.81 |
| | | 72 hr | | | | | | | | |
| | | Stim + | | | | | | | | |
| | | mouse | | | | | | | | |
| | 72 hr | IL-27 | 72 hr | 72 hr | 72 hr | 72 hr | 72 hr | 72 hr | 72 hr | 72 hr |
| mAb | No Add | Only | IgG1 + | IgG2a + | E9633 + | E9630 + | E9631 + | E9629 + | E9632 + | E9518 + |
| conc. | (Avg.) | (Avg.) | IL-27 | IL-27 | IL-27 | IL-27 | IL-27 | IL-27 | IL-27 | IL-27 |
| 0 | 717438.2 | 797.9933 | 1428.98 | 638.99 | 749.06 | 785.51 | 429.81 | 1067.29 | 630.38 | 579.19 |
| 1.875 | | | 760.4 | 766.86 | 1653.77 | 1206.57 | 2419.5 | 3398.88 | 1579.8 | 1457.45 |
| 3.75 | | | 623.72 | 594.29 | 3089.58 | 1376.38 | 2802.65 | 2825.91 | 2607.93 | 1893.65 |
| 7.5 | | | 737.62 | 611.24 | 6265.66 | 2127.66 | 4403.99 | 2810.69 | 6883.83 | 1519.04 |
| 15 | | | 621.98 | 438.73 | 8697.76 | 1627.62 | 5577.57 | 1959.18 | 15117.04 | 2428.29 |
| 30 | | | 832.52 | 1027.1 | 11546.89 | 1629.79 | 4844.64 | 2103.81 | 8902.67 | 2466.65 |
| 60 | | | 634.52 | 556.03 | 13382.28 | 2303.82 | 42444.95 | 1632.5 | 9324.39 | 4058.54 |

### Example 11

Expression vectors encoding human and mouse IL-27RA-Fc fusion proteins were constructed. The fusions comprised the extracellular domain of each IL-27RA fused at its C-terminus (residue 514 of human IL-27RA, SEQ ID NO:3; residue 508 of mouse IL-27RA, SEQ ID NO:17) to the hinge region of the Fc portion of an IgG.gamma.₁ (Ellison et al., Nuc. Acids Res. 10:4071-4079, 1982). The hinge region was modified to replace a cysteine residue with serine to avoid unpaired cysteines upon dimerization of the fusion protein.

Human IL-27RA DNA fragments were prepared from a human IL-27RA cDNA template (Baumgartner et al., U.S. Pat. No. 5,792,850). A 177-bp ApaLI-BglII fragment was prepared by PCR using 1.micro.1 of oligonucleotide primer zc10381 (SEQ ID NO:18) and 4.9.micro.1 of zc10390 (SEQ ID NO:19). The primers were combined with 1.micro.1 of template DNA, 10.micro.1 of 2.5 mM dNTPs (Perkin-Elmer Corp.), 10.micro.1 of 10×buffer (KLENTAQ PCR buffer, Clontech Laboratories, Inc.), 2.micro.1 of DNA polymerase (KLENTAQ; Clontech Laboratories, Inc.), and 71.1.micro.1 H₂O. The reaction was run for 35 cycles of 94° C., 1 minute, 55° C., 1 minute, and 72° C., 2 minutes; followed by a 7-minute incubation at 72° C. The reaction products were extracted with phenol/CHCl₃, precipitated with ethanol, and digested with BglII. The DNA was electrophoresed on an agarose gel, and a 177-bp fragment was electrophoretically eluted from a gel slice, purified by phenol/CHCl₃ extraction, and precipitated with ethanol. A second fragment (1.512 kb) was isolated from the cDNA by digestion with EcoRI and ApaLI.

A human IgG.gamma.₁ clone was isolated from a human fetal liver cDNA library (Clontech Laboratories, Inc.) by PCR using oligonucleotide primers zc10314 (SEQ ID NO:20) and zc10315 (SEQ ID NO:21). The former primer introduced a BglII site into the hinge region (changing the third residue of the hinge region from Lys to Arg) and replaced the fifth residue of the hinge region (Cys) with Ser. PCR was carried out essentially as described above for the IL-27RA reactions. The DNA was digested with EcoRI and XbaI, and a 0.7-kb fragment was recovered by agarose gel electrophoresis, electroelution, phenol/CHCl₃ extraction, and ethanol precipitation. The IgG-encoding fragment and an XbaI-EcoRI linker were ligated into Zem229R (ATCC Accession No. 69447) that had been digested with EcoRI and treated with calf intestinal phosphatase. The resulting plasmid was designated Zem229R IgG.gamma.1#488.

To construct an expression vector for the human IL-27RA-IgG fusion, Zem229R IgG.gamma.1#488 was digested with EcoRI and BglII. The linearized vector was ligated to the two human IL-27RA fragments. The resulting construct was designated hZCYTOR-1/IgG #641.

Mouse IL-27RA DNA fragments were prepared from a full-length mouse IL-27RA cDNA template (Baumgartner et al., ibid.). A 379-bp KpnI-BglII fragment was prepared by PCR essentially as described above using oligonucleotide primers 10382 (SEQ ID NO:22) and 10388 (SEQ ID NO:23). The PCR product was digested with ApaI and gel purified to yield a 46-bp ApaI-BglII fragment. A 1.5-kb fragment was prepared from mZCYTOR-1 T1323 (Baumgartner et al., U.S. Pat. No. 5,792,850) by digestion with EcoRI and ApaI.

The two mouse DNA fragments were ligated to Zem229R IgG.gamma.1#488 that had been digested with EcoRI and BglII. The resulting construct was designated mZYCTOR-1/IgG #632.

The mouse and human IL-27RA/IgG fusion constructs were each transfected into BHK-570 cells by liposome-mediated transfection. Transfectants were cultured in medium containing 1.micro.M methotrexate for 10 days.

Fusion proteins were purified from cell-conditioned media using protein A-Sepharose. Purified protein was used to immunize animals (mice or rabbits) to generate anti-receptor antibodies.

### Example 12

An expression plasmid encoding a soluble human IL27RA with a C-terminal polyhistidine tag was constructed via homologous recombination in yeast with a DNA fragment encoding the extracellular domain of human IL27RA (amino acids 1 to 512 of SEQ ID NO:3) followed by a carboxyl-terminal histidine tag inserted into mammalian expression vector pZMP40.

The indicated fragment of IL27RA cDNA (nucleotides 23-1561 of SEQ ID NO:2) was isolated using PCR. The upstream primer for PCR (zc53405; SEQ ID NO:24) included, from 5′ to 3′ end, 37 by of flanking sequence from the vector and 21 by corresponding to the amino terminus from the open reading frame of IL27RA. The downstream primer (zc52311; SEQ ID NO:25) consisted of, from 5′ to 3′, 50 by of flanking vector sequence, 30 by corresponding to the histidine tag sequence and the last 21 by of the IL27RA extracellular domain coding sequence, nucleotides 1541 to 1561 of SEQ ID NO:2.

The PCR amplification reaction conditions were as follows: 1 cycle, 94° C., 5 minutes; 25 cycles, 94° C., 1 minute, followed by 65° C., 1 minute, followed by 72° C., 1 minute; 1 cycle, 72° C., 5 minutes. Ten μL of each 100 μL PCR reaction mixture was run on a 0.8% low melting temperature agarose gel (SEAPLAQUE GTG) with 1×TBE buffer for analysis. The remaining 90 μL of the PCR reaction mixture and 200 ng of Bgl II-cut pZMP40 were precipitated with the addition of 20 μL 3 M Na Acetate and 500 μL of absolute ethanol, rinsed, dried and resuspended in 10 μL water.

One hundred μL of competent yeast cells (S. cerevisiae) were combined with 10 μL of the DNA mixture from above and transferred to a 0.2-cm electroporation cuvette. The yeast/DNA mixtures were electropulsed at 0.75 kV (5 kV/cm), ∞ ohms, 25 μF. To each cuvette was added 600 μL of 1.2 M sorbitol, and the yeast was plated in two 300 μL aliquots onto two URA-D plates and incubated at 30° C. After about 48 hours, approximately 50 μL packed yeast cells taken from the Ura+yeast transformants of a single plate were treated with B-1,3-glucan laminaripentaohydrolase and b-1,3-glucanase as disclosed in Example 5. This mixture was incubated for 30 minutes at 37° C., then the remainder of the miniprep protocol was performed. The plasmid DNA was eluted twice in 100 μL water and precipitated with 20 μL 3 M Na Acetate and 500 μL absolute ethanol. The pellet was rinsed once with 70% ethanol, air-dried and resuspended in 10 μL water for transformation.

Fifty μL electrocompetent E. coli cells (DH10B, Invitrogen, Carlsbad, Calif.) were transformed with 2 μL yeast DNA. The cells are electropulsed at 1.7 kV, 25 μF and 400 ohms. Following electroporation, 1 mL SOC (2% BACTO Tryptone (Difco, Detroit, Mich.), 0.5% yeast extract (Difco), 10 mM NaCl, 2.5 mM KCl, 10 mM MgCl₂, 10 mM MgSO₄, 20 mM glucose) was plated in 250, 100 and 10 μL aliquots on three LB AMP plates.

Individual clones harboring the correct expression construct for IL27RA CH₆ were identified by restriction digest to verify the presence of the insert and to confirm that the various DNA sequences had been joined correctly to one another. The inserts of positive clones were subjected to sequence analysis. Larger scale plasmid DNA was isolated using a commercially available kit (QIAGEN Maxi kit; QIAGEN Inc.) according to the manufacturer's instruction. The DNA and amino acid sequence are shown in SEQ ID NOS:26 and 27.

Three sets of 200 μg of the IL27RA CH₆ constructs were separately digested with 200 units of PvuI at 37° C. for three hours, precipitated with ethanol, and centrifuged in a 1.5 mL microfuge tube. The supernatant was decanted off the pellet, and the pellet was washed with 300 μL of 70% ethanol and allowed to incubate for 5 minutes at room temperature. The tube was spun in a microfuge for 10 minutes at 14,000 RPM, and the supernatant was decanted off the pellet. The pellet was then resuspended in 750 μL of CHO cell tissue culture medium in a sterile environment, allowed to incubate at 60° C. for 30 minutes, and was allowed to cool to room temperature. Approximately 5×10⁶ CHO cells were pelleted in each of three tubes and resuspended using the DNA-medium solution. The DNA/cell mixtures were placed in a 0.4-cm gap cuvette and electroporated at 950 μF, high capacitance, 300 V. The contents of the cuvettes were then removed, pooled, and diluted to 25 mL with CHO cell tissue culture medium and placed in a 125-mL shake flask. The flask was placed in an incubator on a shaker at 37° C., 6% CO₂ with shaking at 120 RPM.

The CHO cells were subjected to nutrient selection followed by step amplification to 200 nM methotrexate (MTX), then to 1 μM MTX. Tagged protein expression was confirmed by Western blot, and the CHO cell pool was scaled-up for harvests for protein purification.

From the foregoing, it will be appreciated that, although specific embodiments have been described herein for purposes of illustration, various modifications may be made without deviating from the spirit and scope of the invention. Accordingly, the claims are not limited by these exemplary embodiments. All references cited herein are incorporated by reference in their entirety.

1. A polypeptide comprising, from amino terminus to carboxyl terminus, a Zcytor1 fragment with at least 80% sequence identity to SEQ ID NO:3 operably linked to an immunoglobulin Fc fragment, wherein the Fc fragment is a modified Fc fragment wherein amino acid residues at EU index positions 234, 235, and 237 have been substituted to reduce binding to Fc.gamma.RI and amino acid residues at EU index positions 330 and 331 have been substituted to reduce complement fixation, and wherein said polypeptide is optionally glycosylated.

2. The polypeptide of claim 1 wherein said Fc fragment is a human Fc fragment.

3. The polypeptide of claim 1 wherein said Fc fragment is further modified by substitution of a cysteine residue at EU index position 220.

4. The polypeptide of claim 1 wherein said Fc fragment is an Fc5 fragment as shown in FIGS. 1A-1B.

5. The polypeptide of claim 1, wherein said polypeptide consists of amino acid residues 33 to 744 of SEQ ID NO:16.

6. The polypeptide of claim 1, wherein said Zcytor1 fragment consists essentially of an amino acid sequence with at least 80% sequence identity to amino acid residues 33 to 514 of SEQ ID NO:3 with the proviso that residue 41 is a Cys residue, residues 52-54 have a Cys-X-Trp residue sequence, residue 151 is a Trp residue, residue 207 is an Arg residue, and residues 217-221 are a WSXWS domain.

7. The polypeptide of claim 1, wherein said Zcytor1 fragment consists essentially of an amino acid sequence with at least 80% sequence identity to amino acid residues 33 to 235 of SEQ ID NO:3 with the proviso that residue 41 is a Cys residue, residues 52-54 have a Cys-X-Trp residue sequence, residue 151 is a Trp residue, residue 207 is an Arg residue, and residues 217-221 are a WSXWS domain.

8. A polypeptide consisting essentially of, from amino terminus to carboxyl terminus, a Zcytor1 fragment with at least 80% sequence identity to residues 33 to 514 of SEQ ID NO:3 operably linked to an immunoglobulin Fc fragment, wherein the Fe fragment is a modified Fc fragment wherein amino acid residues at EU index positions 234, 235, and 237 have been substituted to reduce binding to Fc.gamma.RI and amino acid residues at EU index positions 330 and 331 have been substituted to reduce complement fixation, and wherein said polypeptide is optionally glycosylated.

9. The polypeptide of claim 8 wherein said Fc fragment is a human Fc fragment.

10. The polypeptide of claim 8 wherein said Fc fragment is further modified by substitution of a cysteine residue at EU index position 220.

11. The polypeptide of claim 8 wherein said Fc fragment is an Fc5 fragment as shown in FIGS. 1A-1B.

12. The polypeptide of claim 8 wherein said Zcytor1 fragment is at least 80% identical to residues 33 to 235 of SEQ ID NO:3 and said Fc fragment is an Fc5 fragment with an amino acid sequence as shown in FIGS. 1A-1B.

13. A dimeric protein consisting of two polypeptides joined by a disulfide bond, each of said polypeptides independently comprising, from amino terminus to carboxyl terminus, a Zcytor1 fragment with at least 80% sequence identity to SEQ ID NO:3 operably linked to an immunoglobulin Fc fragment, wherein said immunoglobulin Fc fragment is a modified Fc fragment wherein the amino acid residues at EU index positions 234, 235, and 237 have been substituted to reduce binding to Fc.gamma.RI and the amino acid residues at EU index positions 330 and 331 have been substituted to reduce complement fixation, wherein the protein binds IL-27 and wherein said dimeric protein is optionally glycosylated.

14. The protein of claim 13 wherein said Zcytor1 fragment of at least one of said two polypeptides consists of an amino acid sequence with at least 80% sequence identity to residues 33 to 514 of SEQ ID NO:3.

15. The protein of claim 13 wherein the Fc fragment of at least one of said two polypeptides is a human Fc fragment.

16. The protein of claim 13 wherein the Fc fragment of at least one of said two polypeptides is further modified by substitution of a cysteine residue at EU index position 220.

17. The protein of claim 13 wherein the Fc fragment of at least one of said two polypeptides is an Fc5 fragment as shown in FIGS. 1A-1B.

18. The protein of claim 13, wherein at least one of said two polypeptides consists of amino acid residues 33 to 744 of SEQ ID NO:16.

19-20. (canceled)

21. A polynucleotide consisting essentially of a polynucleotide sequence encoding a polypeptide of claim 1.

22. The polynucleotide of claim 21 wherein said polynucleotide sequence is cloned into an expression vector.

23. The polynucleotide of claim 21 wherein said polynucleotide sequence is at least 80% identical to SEQ ID NO:2.

24. A polynucleotide consisting essentially of a polynucleotide sequence encoding a polypeptide of claim 8.

25. A polynucleotide consisting essentially of a polynucleotide sequence encoding a polypeptide of claim 13.

26. A pharmaceutical composition comprising the polypeptide of claim 1.
